(19) United States
(12) Patent Application Publication
(10) Pub. No.: US 2026/0235531 A1
(43) Pub. Date: Aug. 13, 2026

(54) RESIN COMPONENT APPEARANCE INSPECTION DEVICE AND RESIN COMPONENT APPEARANCE INSPECTION METHOD

(71) Applicant: Mitsubishi Electric Corporation, Chiyoda-ku, Tokyo (JP)

(72) Inventors: Michinobu FUJIWARA, Chiyoda-ku, Tokyo (JP); Yuka YOKOBORI, Chiyoda-ku, Tokyo (JP); Kazuyuki SHIBATA, Chiyoda-ku, Tokyo (JP)

(73) Assignee: Mitsubishi Electric Corporation, Chiyoda-ku, Tokyo (JP)

(21) Appl. No.: 19/153,473

(22) PCT Filed: Feb. 1, 2024
(86) PCT No.: PCT/JP2024/003267

§ 371 Date: Aug. 4, 2025

(30) Foreign Application Priority Data
Mar. 22, 2023 (JP) 2023-045038

(51) Int. Cl. G01N 21/95; G01N 33/44

(52) U.S. Cl. CPC G01N 21/95; G01N 33/442

(57) ABSTRACT

A resin component appearance inspection device includes: a component retention mechanism which retains a resin component which is an inspection target; a camera which takes an image of the resin component; an illumination which irradiates the resin component; and an image processing device which performs calculation processing on the image taken by the camera, wherein the resin component is opaque and lustrous, and the image processing device performs calculation processing on the image taken by the camera, to detect a defective part of a surface of the resin component, and quantifies an appearance quality of the resin component.

### CROSS-REFERENCE TO RELATED APPLICATIONS

This application is the United States National Phase under 35 U.S.C. § 371 of PCT International Patent Application No. PCT/JP2024/003267, filed on Feb. 1, 2024, which claims the benefit of Japan Patent Application No. 2023-045038, filed on Mar. 22, 2023, the disclosures of which are hereby incorporated by reference herein in their entireties.

### TECHNICAL FIELD

The present disclosure relates to a resin component appearance inspection device and a resin component appearance inspection method.

### BACKGROUND ART

Defects of resin components include shape faults such as a warp, a twist, and a sink mark, thermally caused faults such as a black dot and a foreign material, mold faults such as a flaw and a burr, and appearance faults such as a flow mark and a silver streak. Among these, appearance inspection for a flow mark and the like is generally performed through visual inspection by an inspector. In the visual inspection, an evaluation method is based on a personal skill and depends on determination by an inspector, and thus numerical quantification cannot be performed. Therefore, it is difficult to keep the appearance quality constant.

For avoiding dependence on a personal skill, disclosed is a defect detection classification system that uses machine learning and includes an acquisition section which acquires an image and a detection classification section which determines presence/absence, a position, and a type of a defect (for example, Patent Document 1).

In addition, disclosed is an inspection system that includes an inspection camera which takes an image of an inspection area including a part of an inspection target, a detection camera which takes an image of a detection area larger than the inspection area, a stage on which the inspection target is placed, and an adjustment mechanism which adjusts relative positions of the stage and the inspection camera on the basis of the image of the detection area (for example, Patent Document 2).

### CITATION LIST

### Patent Document

Patent Document 1: Japanese Laid-Open Patent Publication No. 2021-139789
Patent Document 2: WO2020/121977

### SUMMARY OF THE INVENTION

### Problem to be Solved by the Invention

However, in the system in Patent Document 1, since machine learning is used, a large amount of training data is needed. In addition, the systems in Patent Documents 1 and 2 cannot detect a defect unless the defect is a high-contrast appearance defect such as stain on an outer surface or an appearance defect due to a macroscopic recess/projection.

The present disclosure has been made to solve the above problems, and an object of the present disclosure is to provide a resin component appearance inspection device and a resin component appearance inspection method that can perform quantification even for an appearance defect that has a low contrast and is not due to a macroscopic recess/projection, while the number of images needed for inspection is small.

### Means to Solve the Problem

A resin component appearance inspection device according to the present disclosure includes a component retention mechanism which retains a resin component which is an inspection target; a camera which takes an image of the resin component; an illumination which irradiates the resin component; and an image processing device which performs calculation processing on the image taken by the camera. The resin component is opaque and lustrous. The image processing device performs calculation processing on the image taken by the camera, to detect a defective part of a surface of the resin component, and quantifies an appearance quality of the resin component.

A resin component appearance inspection method according to the present disclosure is performed using a component retention mechanism which retains a resin component which is an inspection target, a camera which takes an image of the resin component, an illumination which irradiates the resin component, and an image processing device which performs calculation processing on the image taken by the camera, the method including an image acquisition step of acquiring the image taken by the camera, into the image processing device; a trimming step of trimming a defective part of a surface of the resin component; a smoothing step of smoothing an image generated in the trimming step; a difference image generation step of generating an image representing a difference between the image generated in the trimming step and an image generated in the smoothing step; a binarization step of binarizing the image generated in the difference image generation step; a contour extraction step of extracting a contour of the defective part; and a quantitative value calculation step of calculating a pixel ratio of the defective part to perform quantification. A resin component appearance inspection method according to the present disclosure is a resin component appearance inspection method in which, using an injection molding device for performing injection molding of a resin component which is an inspection target, appearance quality inspection for the resin component is performed. In a state in which a camera which takes an image of the resin component and an illumination which irradiates the resin component are placed at a platen or a mold to be used for resin molding of the resin component, the method is performed using an image processing device which performs calculation processing on the image taken by the camera, a movable blackout screen, and a taking-out machine which takes the resin component out of the mold and moves the resin component, the method including a position adjustment step of adjusting positions of the camera and the illumination; a consecutive molding start step of starting consecutive molding of the resin component; a resin loading step of loading resin into the mold; a first blackout screen movement step of moving the blackout screen close to the mold; a mold opening step of opening the mold after loading of the resin in the resin loading step is completed; an imaging step of emitting illumination light to the resin component by the illumination and taking an image of the resin component by the camera; a second blackout screen movement step of moving the blackout screen to outside of the mold; a chucking step of moving the taking-out machine close to the resin component and chucking the resin component; an image acquisition step of acquiring the image of the resin component taken by the camera, into the image processing device; an image processing step of performing image processing on the acquired image and calculating a quantitative value of an appearance quality of the resin component; a quantitative value output step of outputting the quantitative value of the appearance quality of the resin component calculated in the image processing step; a quality determination step of comparing the appearance quality of the resin component outputted in the quantitative value output step with a predetermined required quality, and determining whether or not the required quality is satisfied; a proper product processing step of, in a case where it is determined that the required quality is satisfied in the quality determination step, moving the resin component to a proper product place by the taking-out machine and returning to the resin loading step; a defective product processing step of, in a case where it is determined that the required quality is not satisfied in the quality determination step, moving the resin component to a defective product place by the taking-out machine; a defect frequency determination step of determining whether or not a defective product has been produced consecutively a predetermined threshold number of times or more, and in a case where a defective product has not been produced consecutively the predetermined threshold number of times or more, returning to the resin loading step, and in a case where a defective product has been produced consecutively the predetermined threshold number of times or more, proceeding to an alert output step; and the alert output step of outputting a consecutive defect alert and stopping a consecutive molding process.

### Effect of the Invention

With the resin component appearance inspection device according to the present disclosure, it becomes possible to provide a resin component appearance inspection device that can perform quantification even for an appearance defect that has a low contrast and is not due to a macroscopic recess/projection, while the number of images needed for inspection is small. With the resin component appearance inspection method according to the present disclosure, it becomes possible to provide a resin component appearance inspection method that can perform quantification even for an appearance defect that has a low contrast and is not due to a macroscopic recess/projection, while the number of images needed for inspection is small.

### BRIEF DESCRIPTION OF THE DRAWINGS

FIG. 1 is an isometric view of an entire resin component appearance inspection device according to embodiment 1.

FIG. 2 shows a structure of the resin component appearance inspection device according to embodiment 1.

FIG. 3A conceptually shows an illumination and reflection light according to embodiment 1, and FIG. 3B illustrates reflection light according to embodiment 1.

FIG. 4 illustrates an incidence angle of the illumination and a detection angle of a camera according to embodiment 1.

FIG. 5 illustrates an appropriate detection angle range according to embodiment 1.

FIG. 6A illustrates an actual measurement result regarding the detection angle according to embodiment 1, and FIG. 6B illustrates an actual measurement result regarding the detection angle according to embodiment 1.

FIG. 7 illustrates an example of an irregular reflection application image according to embodiment 1.

FIG. 8 illustrates detection angle appropriateness determination according to embodiment 1.

FIG. 9A illustrates a luminance distribution characteristic of the illumination according to embodiment 1, FIG. 9B illustrates a luminance distribution characteristic of the illumination according to embodiment 1, and FIG. 9C illustrates a luminance distribution characteristic of the illumination according to embodiment 1.

FIG. 10 illustrates a length requirement for an illumination light emitting portion according to embodiment 1.

FIG. 11 is a flowchart of a resin component appearance inspection method according to embodiment 1.

FIG. 12 is a flowchart of image processing in resin component appearance inspection according to embodiment 1.

FIG. 13A shows an example of a processed image in resin component appearance inspection according to embodiment 1, FIG. 13B is an example of a processed image in resin component appearance inspection according to embodiment 1, FIG. 13C is an example of a processed image in resin component appearance inspection according to embodiment 1, FIG. 13D is an example of a processed image in resin component appearance inspection according to embodiment 1, FIG. 13E is an example of a processed image in resin component appearance inspection according to embodiment 1, and FIG. 13F is an example of a processed image in resin component appearance inspection according to embodiment 1.

FIG. 14 shows examples of processed images of a defective component in resin component appearance inspection according to embodiment 1.

FIG. 15 shows examples of processed images of a defective component in resin component appearance inspection according to embodiment 1.

FIG. 16 shows a structure of a resin component appearance inspection device according to embodiment 2.

FIG. 17 is a flowchart showing a resin component appearance inspection method according to embodiment 2.

FIG. 18 shows a structure of a resin component appearance inspection device according to embodiment 3.

FIG. 19 is a flowchart of a resin component appearance inspection method according to embodiment 3.

FIG. 20 is a system configuration diagram of a resin component appearance inspection device according to embodiment 4.

FIG. 21 is a system configuration diagram of the resin component appearance inspection device according to embodiment 4.

FIG. 22 is a function block diagram of devices involved in an injection molding process according to embodiment 4.

FIG. 23A illustrates the injection molding process according to embodiment 4, and FIG. 23B illustrates the injection molding process according to embodiment 4.

FIG. 24A illustrates the injection molding process according to embodiment 4, and FIG. 24B illustrates the injection molding process according to embodiment 4.

FIG. 25 is a flowchart of a resin component appearance inspection method according to embodiment 4.

FIG. 26 is a flowchart of the resin component appearance inspection method according to embodiment 4.

FIG. 27 is a hardware configuration example of an image processing device and a control device.

### DESCRIPTION OF EMBODIMENTS

### Embodiment 1

Embodiment 1 relates to a resin component appearance inspection method and a resin component appearance inspection device including a component retention mechanism which retains a resin component which is an inspection target; a camera which takes an image of the resin component; an illumination which irradiates the resin component; and an image processing device which performs calculation processing on the image taken by the camera, wherein the image processing device performs processing on the image taken by the camera, to detect a defective part of a surface of the resin component, and quantifies an appearance quality of the resin component.

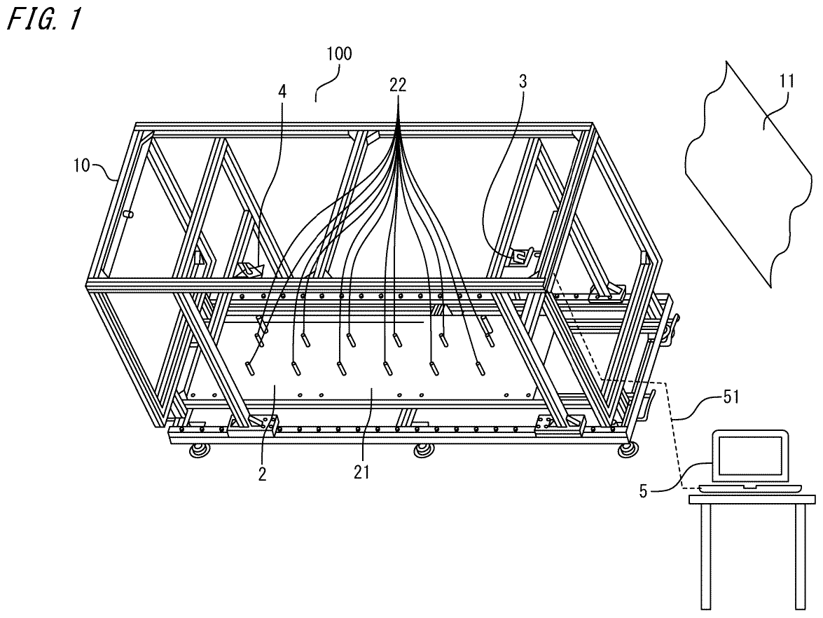

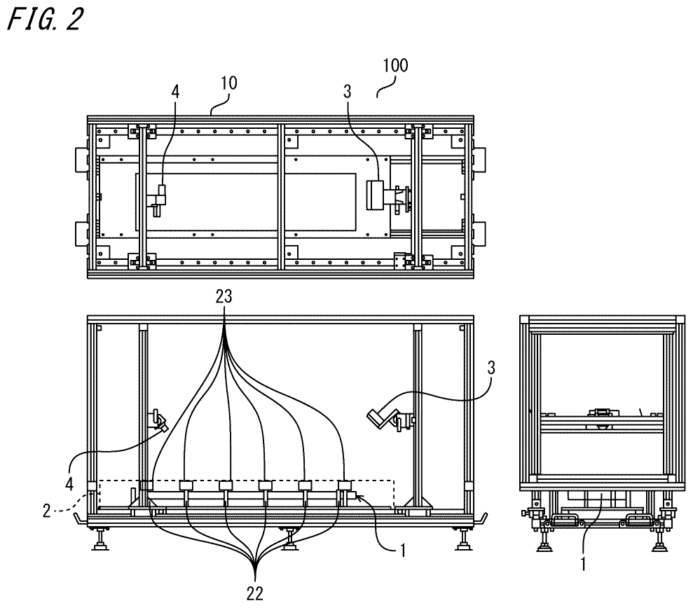

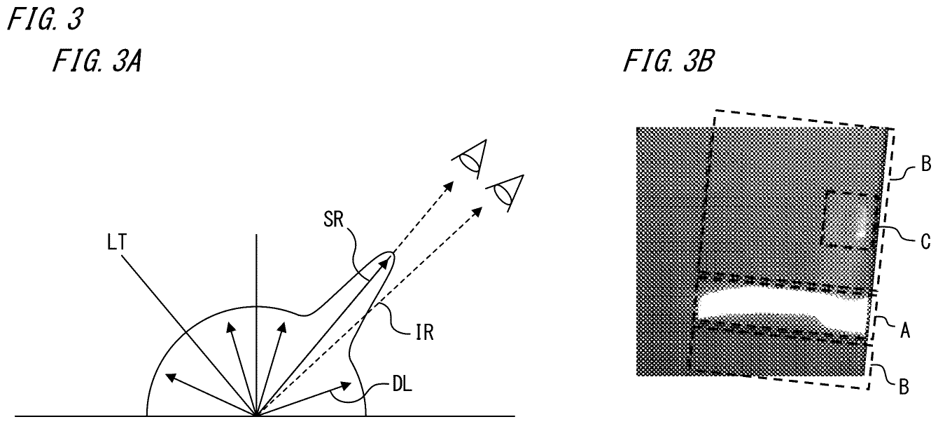

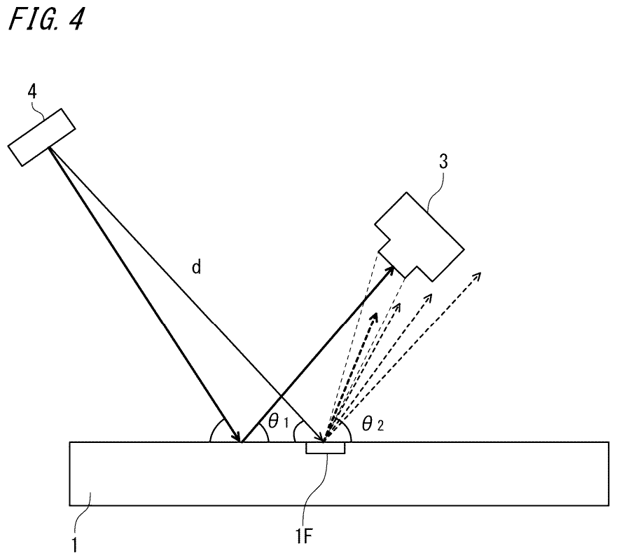

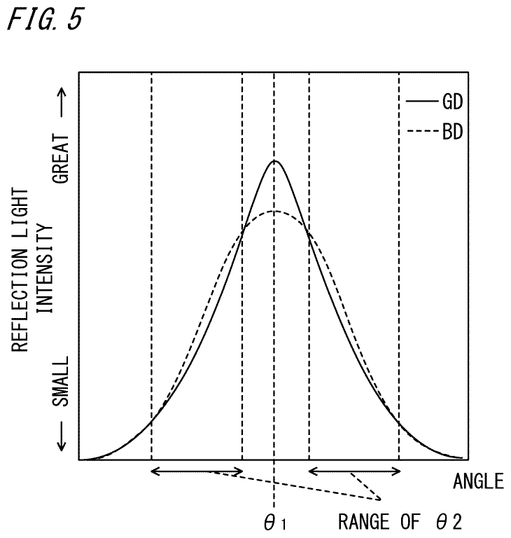

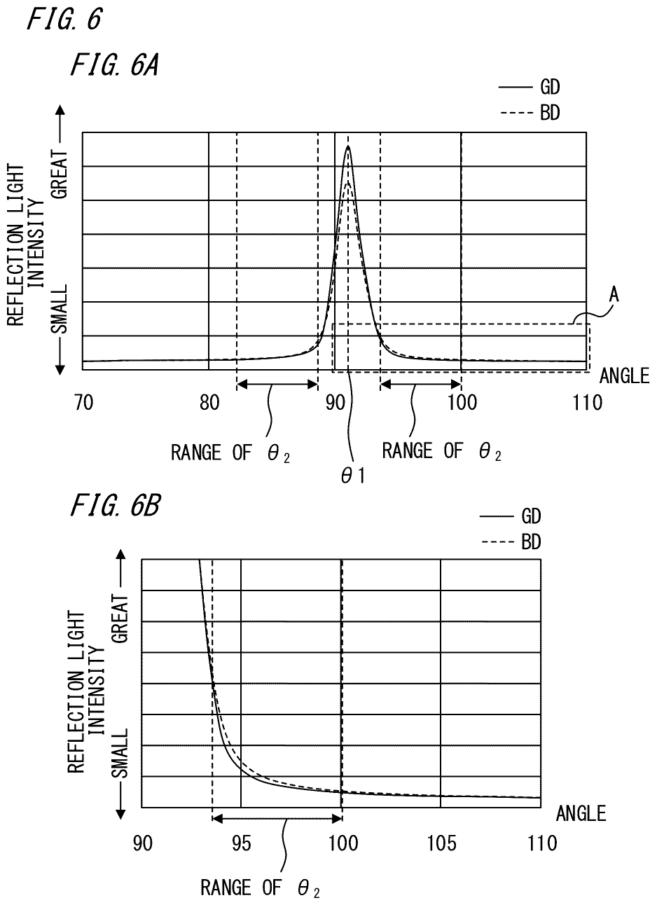

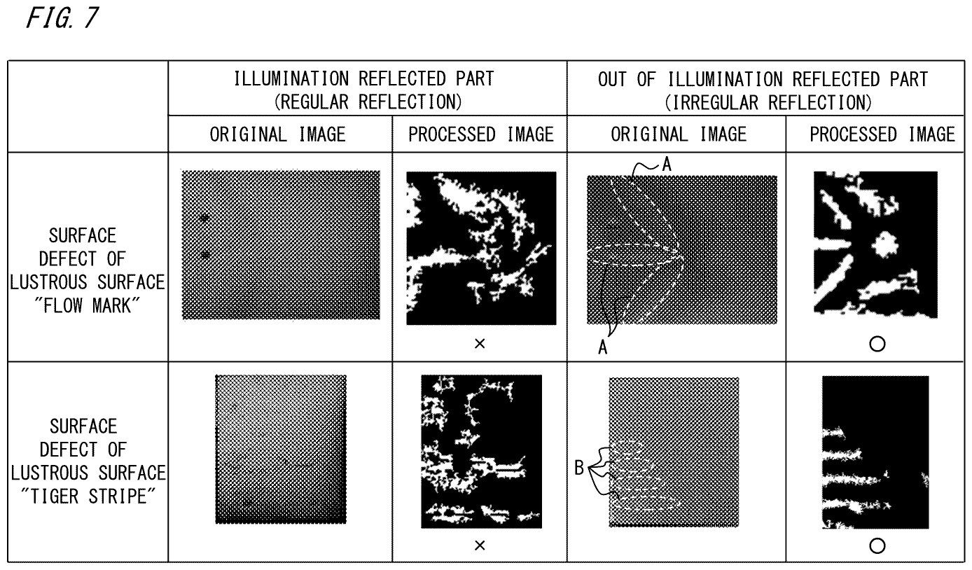

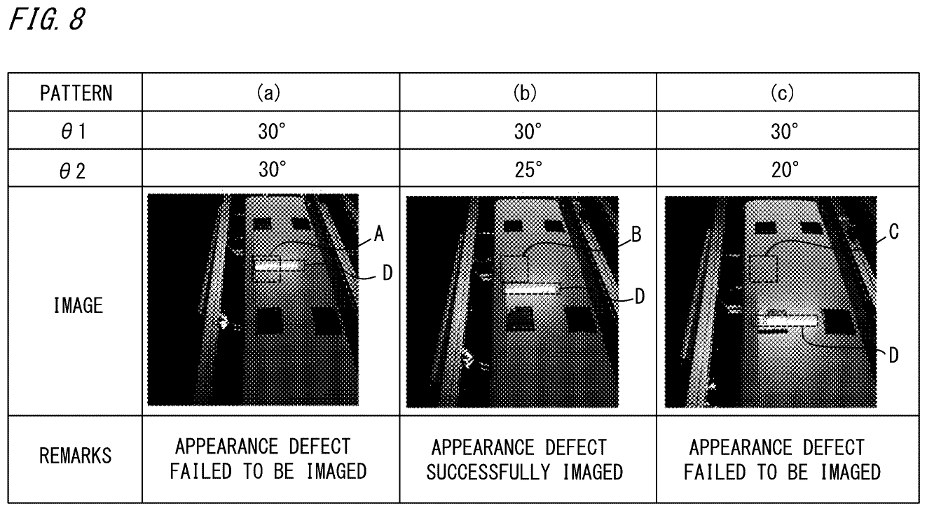

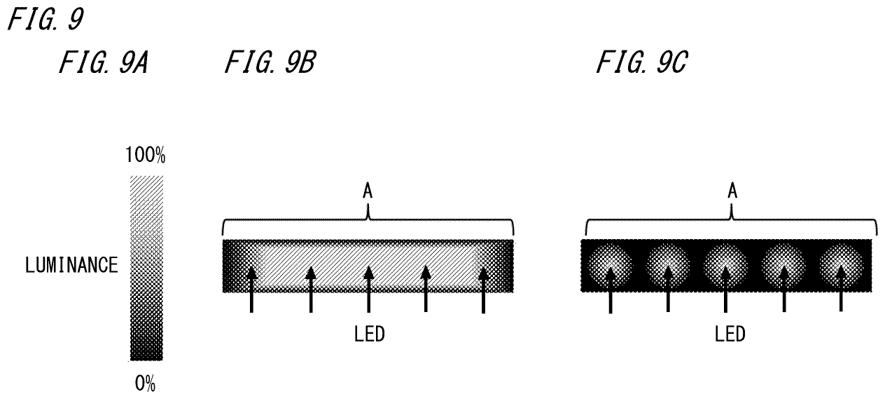

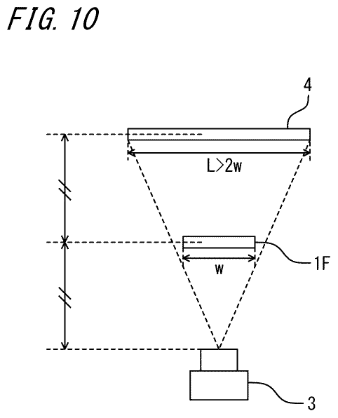

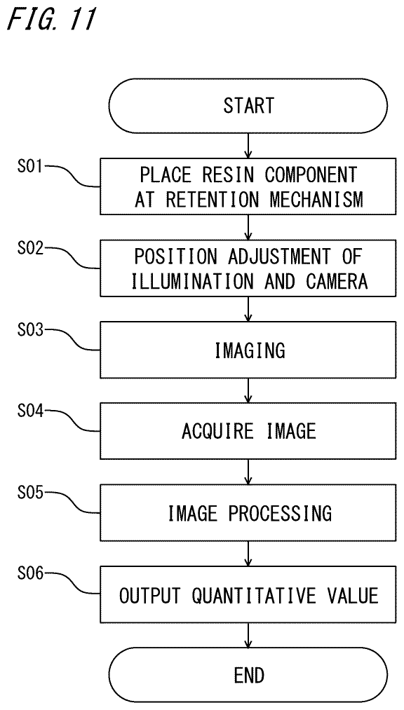

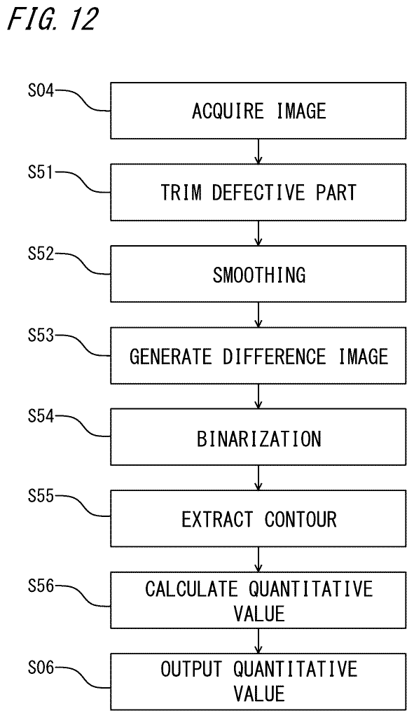

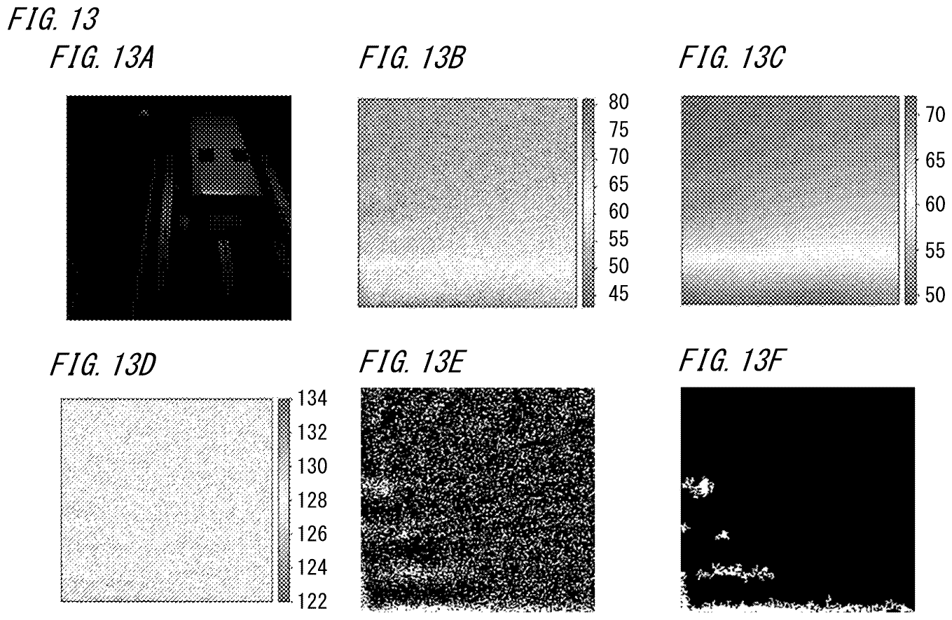

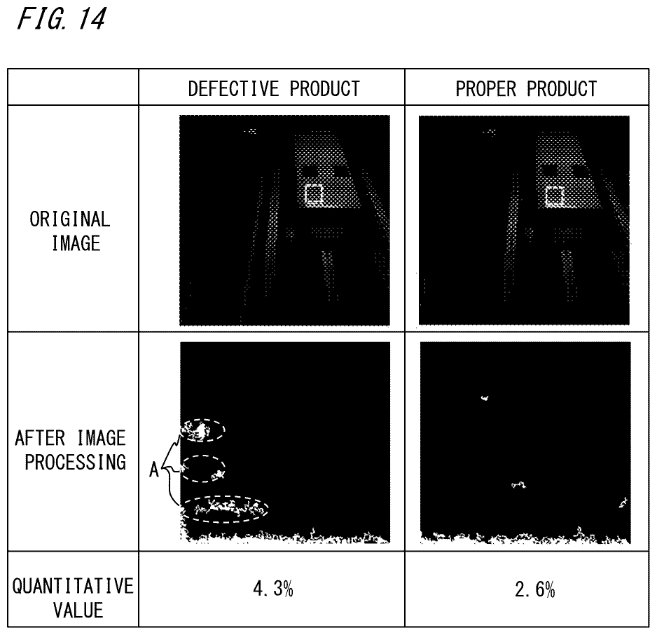

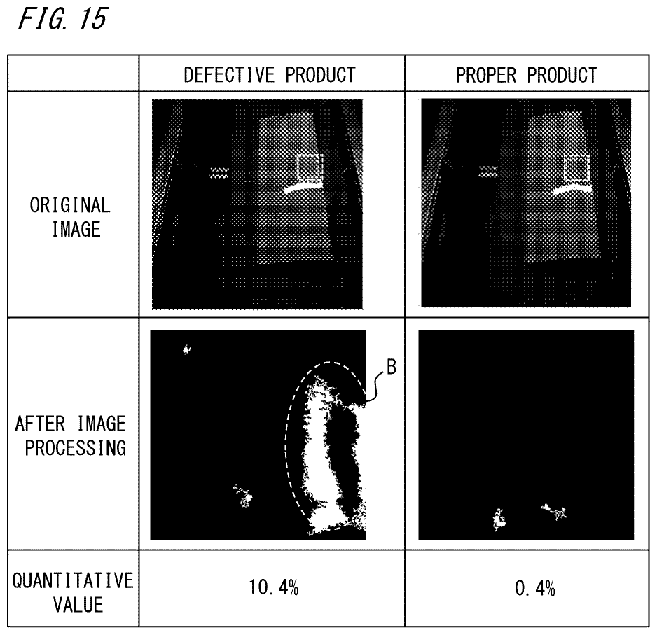

Hereinafter, a resin component appearance inspection device and a resin component appearance inspection method according to embodiment 1 will be described with reference to FIG. 1 which is an isometric view of the entire resin component appearance inspection device, FIG. 2 which shows a structure of the resin component appearance inspection device, FIG. 3A and FIG. 3B which conceptually show an illumination and reflection light, FIG. 4 illustrates an incidence angle of the illumination and a detection angle of the camera, FIG. 5 which illustrates an appropriate detection angle range, FIG. 6A and FIG. 6B which illustrate actual measurement results regarding the detection angle, FIG. 7 which illustrates an example of an irregular reflection application image, FIG. 8 which illustrates detection angle appropriateness determination, FIG. 9A, FIG. 9B, and FIG. 9C which illustrate a luminance distribution characteristic of the illumination, FIG. 10 which illustrates a length requirement for an illumination light emitting portion, FIG. 11 which is a flowchart of a resin component appearance inspection method, FIG. 12 which is a flowchart of image processing in resin component appearance inspection, FIG. 13A to FIG. 13F which show examples of processed images in resin component appearance inspection, and FIG. 14 and FIG. 15 which show examples of processed images of defective components in resin component appearance inspection.

In the drawings, the same or corresponding parts are denoted by the same reference characters.

First, the entire structure of a resin component appearance inspection device 100 according to embodiment 1 will be described with reference to FIG. 1 which is an isometric view of the entire inspection device and FIG. 2 which shows the structure of the inspection device.

The resin component appearance inspection device 100 includes, as main constituent parts, a resin component 1 which is an inspection target, a component retention mechanism 2 which retains the resin component 1, a camera 3 and an illumination 4 for taking an image of the resin component 1, an image processing device 5 which processes the taken image, a framework 10 which is a frame structure of the entire inspection device, and a blackout screen 11 which blocks ambient light.

In embodiment 1, it is assumed that appearance inspection for the resin component 1 is performed through visual recognition in advance to specify a part where an appearance defect is likely to arise, and presence/absence of a defect at the specified part is inspected. Therefore, in performing appearance inspection, the distances and the angles of the camera 3 and the illumination 4 relative to the resin component 1 are adjusted so that inspection for the defective part specified in advance can be performed with high accuracy.

The resin component 1 which is an inspection target is a component molded by a mold, and if the resin component 1 has a warp, resin component appearance inspection described later cannot be performed accurately. Therefore, it is assumed that, if the resin component 1 has a warp, the warp is corrected before inspection.

The component retention mechanism 2 retains the resin component 1 in a state of being lifted up from a floor, in order to inspect the appearance of the resin component 1.

The camera 3 takes an image for performing appearance inspection for the resin component 1, and the illumination 4 emits illumination light needed for the camera 3 to take an image. The camera 3 and the illumination 4 are attached to the framework 10 and are fixed so as to keep appropriate distances and angles relative to the resin component 1 which is an inspection target.

A fastener for attaching the camera 3 and the illumination 4 to the framework 10 and adjusting the angles thereof is needed, but such a fastener is known and therefore the description thereof is omitted.

The camera 3 and the illumination 4 are connected to the image processing device 5 via a cable 51, and the image processing device 5 performs an operation for lighting up or turning off the illumination 4, and an operation for taking an image of the resin component 1 by the camera 3. Lighting up and extinction of the illumination 4 may be controlled by another power supply device or the like, instead of being controlled by the image processing device 5, or the illumination 4 may be kept lit up during inspection. Instead of the image processing device 5 controlling imaging of the camera 3, imaging may be performed using a timer imaging function of a commercially available compact digital camera or the like.

In FIG. 1, a cable connecting the image processing device 5 and the illumination 4 is not shown.

Next, each of the constituent parts will be sequentially described.

First, the resin component 1 will be described. The resin component 1 has a flat plate shape, and an elongated component having dimensions of approximately 300 mm to 900 mm in length and 20 mm to 300 mm in width is also assumed to be an inspection target. The resin component 1 is opaque and lustrous.

The back surface of the resin component 1 may have a rib and such a resin component can also be inspected.

Next, the component retention mechanism 2 will be described.

The component retention mechanism 2 includes an aluminum plate 21, knurled bolts 22, and magnets 23.

The resin component 1 is fixed by being held between the head of the knurled bolt 22 and the magnet 23.

A tapped hole (or threaded insert) for fixing the knurled bolt 22 is provided in the aluminum plate 21, and the knurled bolt 22 made of iron is inserted and fixed therein. The resin component 1 is placed at the top of the knurled bolt 22, the magnet 23 is put on the resin component 1, and the resin component 1 is fixed by being held between the magnet 23 and the head of the knurled bolt 22. With this structure, the resin component 1 is retained in a state of being lifted up from a floor.

The position of the knurled bolt 22 is set at such a position that the knurled bolt 22 and a rib of the resin component 1 do not interfere with each other.

By configuring the component retention mechanism 2 as described above, it is possible to ensure reproducibility of a retention position for the resin component 1 having a warp and the resin component 1 having a projection such as a rib.

In a case of handling a plurality of resin components 1, a plurality of aluminum plates 21 may be provided correspondingly to the respective resin components 1, or the resin components 1 may be gathered up at one aluminum plate 21. In a case of gathering up the resin components 1 at one aluminum plate 21, positions where ribs of the resin components 1 do not interfere are set, and then tapped holes for all the target resin components 1 are provided in one aluminum plate 21.

The reason for using the knurled bolt 22 instead of a dowel pin is as follows. In a case of attaching the dowel pin by interference fit or transition fit, it is difficult to extract and insert the dowel pin. In a case of attaching the dowel pin by clearance fit, if the resin component 1 has a warp, the dowel pin might come off the hole of the aluminum plate 21.

First, before specifically describing the appearance inspection method, the definitions of terms used here will be described, to describe basic concepts of the illumination and reflection light.

General definitions of basic terms regarding optics are described below.
(1) Specular reflection: Reflection that has an incidence angle and a reflection angle equal to each other and occurs at the interface between two materials.
(2) Regular reflection: A type of specular reflection that occurs on a surface having almost no irregularities. This reflection is characterized by exhibiting a mirrored virtual image.
(3) Irregular reflection: A type of specular reflection that occurs on a rough surface having irregularities
(4) Diffuse reflection: An antonym of specular reflection. This reflection is characterized by reflecting light in various directions at approximately equal intensities without depending on reflection angles. This reflection is due to scattering, transmission, and multiple reflection occurring on a slightly inner side of the interface of a material.

Next, the “regular reflection” and the “irregular reflection” which are important in the later description will be described using specific examples.

In a case where an illumination irradiates a lustrous resin molded product, the appearance of the illumination appears reflected on the surface of the molded product. This reflection is referred to as regular reflection, in the present disclosure. Meanwhile, since the surface of the molded product at both of a proper-appearance part and a defective-appearance part is not a perfect mirror surface, reflection occurs also at angles slightly different from the incidence angle. This reflection is referred to as irregular reflection, in the present disclosure.

A case where an illumination irradiates a lustrous resin molded product having an appearance defect and an image thereof is taken by a camera will be described with reference to FIG. 3A and FIG. 3B.

In FIG. 3A, LT denotes light, SR denotes regular reflection light, IR denotes irregular reflection light, and DL denotes diffused light.

In FIG. 3B, A indicates a part where the illumination appears reflected by regular reflection.

B indicates an area corresponding to irregular reflection outside the part where the illumination appears reflected, that is, indicates an area around the part where the illumination appears reflected. The area B shines dimly and becomes darker as becoming farther from the part where the illumination appears reflected.

C indicates a part having an appearance defect. This part appears a little bright in the area B outside the part where the illumination appears reflected.

The phenomenon described above is due to the fact that the angle distributions of regular reflection light intensities and irregular reflection light intensities are different between the proper-appearance part and the defective-appearance part.

Next, an appropriate relationship between the incidence angle of an illumination and the detection angle of a camera will be described with reference to FIG. 4.

In FIG. 4, dotted-line arrows represent “regular reflection light having an angle distribution”.

An angle formed by the illumination 4 and a defective part 1F of the surface of the resin component 1 is defined as an incidence angle θ1, and the incidence angle θ1 is set at 25° to 35°. An angle formed by the camera 3 and the defective part 1F of the surface of the resin component 1 is defined as a detection angle θ2, and the detection angle θ2 is provided with a difference of about 3° to 10° upward or downward from the incidence angle θ1. By providing a difference between the incidence angle θ1 and the detection angle θ2, it is possible to perform detection at an angle at which a difference in reflection light intensities is exhibited between a proper part and the defective part 1F, among rays of the regular reflection light having an angle distribution. The reason for providing a difference between the incidence angle θ1 and the detection angle θ2 will be described later.

The distance (d in FIG. 4) between the illumination 4 and the defective part 1F of the surface of the resin component 1 is set at 500 mm or greater, whereby straightness of a light ray can be ensured.

The defective part 1F is a defective-appearance part of the resin component 1 which is an inspection target, and a shape fault, a mold fault, and the like are not inspection targets. The “defective part of the surface of the resin component” may be referred to as a “defective part of the resin component”.

Next, the reason for providing a difference between the incidence angle and the detection angle will be described with reference to FIG. 5 which illustrates an appropriate detection angle range. In FIG. 5, GD corresponding to a solid line indicates a “proper part”, and BD corresponding to a dotted line indicates a “defective part”.

Function curves in FIG. 5 conceptually show bidirectional scattering distribution functions (BSDF) obtained by measuring the proper part and the defective part 1F of the resin component 1 using a scattering measurement device. FIG. 5 is a graph in which the concept is emphasized so as to clarify the difference, whereas FIG. 6A and FIG. 6B show an actual measurement result.

FIG. 6A shows an actual measurement result corresponding to FIG. 5. FIG. 6B is an enlarged view of a part A in FIG. 6A.

As shown in FIG. 6B, a slight light intensity difference arises in a certain angle range.

For both of the proper part and the defective part 1F, an angle at which the reflection light intensity is maximized is an angle equal to the incidence angle θ1, and the reflection light intensity is reduced as becoming farther from the incidence angle θ1.

For the defective part 1F, the angle distribution of reflection light intensities is wider than for the proper part, and in a “set range of the detection angle θ2” in the graphs, the reflection light intensity is greater for the defective part 1F. On the other hand, in the vicinity of the incidence angle θ1, the reflection light intensity is greater for the proper part, but such an angle range is narrower than the “set range of the detection angle θ2”. Therefore, it is difficult to adjust the position of the camera 3 in the vicinity of the incidence angle θ1.

Accordingly, the detection angle θ2 of the camera 3 is set in the “set range of the detection angle θ2” in which the reflection light intensity is greater for the defective part 1F. Therefore, it is necessary to provide the detection angle θ2 of the camera 3 with a difference of about 3° to 10° upward or downward from the incidence angle θ1.

In a case of not providing a difference between the detection angle and the incidence angle, that is, in a case of performing image processing by trimming a part where the illumination appears reflected (regular reflection), it is impossible to detect an appearance defect that has a low contrast and is not due to a macroscopic recess/projection, such as a flow mark defect or a tiger stripe defect of a resin component.

Actual examples will be described with reference to FIG. 7. FIG. 7 illustrates a result of defect determination processing based on regular reflection (a part where the illumination appears reflected) and irregular reflection (a part outside the part where the illumination appears reflected), regarding a “flow mark” and a “tiger stripe” which are surface defects of a lustrous surface. At processed images, “o” indicates that the defect can be detected, and “x” indicates that the defect cannot be detected.

In a case of regular reflection, neither a flow mark defect nor a tiger stripe defect can be detected, whereas in a case of irregular reflection, they can be detected.

For a flaw, chipping, stain, and the like among appearance defects of a resin component, a non-regular reflection part (dark part) arises in the regular reflection light and thus the defect can be detected. However, with the regular reflection light, it is impossible to detect an appearance defect that is not due to a macroscopic recess/projection, such as a flow mark defect or a tiger stripe defect.

As described above, using the fact that angle distributions of light intensities of regular reflection and light intensities of irregular reflection are different between the proper part and the defective part, the incidence angle is set at 35 degrees to 55 degrees and the detection angle is provided with an angle difference of 3 degrees to 10 degrees relative to the incidence angle. Then, by trimming in image processing described later, a regular reflection light part of specular reflection light is removed and an irregular reflection light part is extracted. Thus, it is possible to detect a flow mark defect and a tiger stripe defect of the resin component 1 which are appearance defects that have a low contrast and are not due to a macroscopic recess/projection.

Next, a method for determining whether or not the detection angle is appropriate will be described with reference to FIG. 8 which illustrates detection angle appropriateness determination.

In FIG. 8, A indicates that “a defective-appearance part cannot be imaged”, B indicates that “a defective-appearance part can be imaged”, C indicates that “a defective-appearance part cannot be imaged”, and D indicates “a part where an illumination appears reflected”.

In FIG. 8, in a taken image for a placement pattern (a) (incidence angle θ1=30°, detection angle θ2=30°), the part where the illumination 4 appears reflected overlaps the defective part of the resin component, and therefore it is determined that the appearance defect cannot be imaged. In a taken image for a placement pattern (c) (incidence angle θ1=30°, detection angle θ2=20°), the part where the illumination 4 appears reflected is far from the defective part of the resin component, and therefore it is determined that the appearance defect cannot be imaged.

On the other hand, in a placement pattern (b) (incidence angle θ1=30°, detection angle θ2=25°) in which the detection angle θ2 is middle, it can be confirmed that the defective part of the resin component can be imaged, and therefore it is determined that the appearance defect can be detected.

In the above determination method, in a case of corresponding to the placement pattern (a) or the placement pattern (c), the positional relationship between the illumination 4 and the resin component 1 and the positional relationship between the camera 3 and the resin component 1 are adjusted so as to correspond to the placement pattern (b).

Next, requirements imposed on the illumination 4 will be described with reference to FIG. 9A to FIG. 9C which illustrate a luminance distribution characteristic of the illumination. FIG. 9A shows luminance of 0% to 100%, FIG. 9B shows an example of a uniform luminance distribution, and FIG. 9C shows an example of a nonuniform luminance distribution. The illumination 4 used here is an illumination that has a bar shape, emits light in a diffusing manner, and is a surface light emission type in which point light sources do not visually appear. In addition, the luminance distribution characteristic in the long-axis direction is highly uniform, and the length of a light emitting portion is not smaller than two times the length of the defective part 1F of the resin component 1.

FIG. 9B and FIG. 9C show examples in which the luminance distribution characteristic in the long-axis direction of the illumination is highly uniform and less uniform. In FIG. 9C, the luminance is high only around an LED element. In contrast, in FIG. 9B, the luminance is uniform over the entire range in the long-axis direction of LEDs, except for ends.

Next, the relationship of the length of the light emitting portion of the illumination 4 and the length of the defective part 1F of the resin component 1 will be described with reference to FIG. 10 which illustrates length requirements for the illumination light emitting portion.

In order to irradiate the entire defective part 1F of the resin component 1 with uniform light, the length (L) of the light emitting portion of the illumination 4 is set to be not smaller than two times the length (w) of the defective part 1F of the resin component 1.

In a case where the length (L) is smaller than two times the length (w), incident light intensities are small only at ends of the defective part 1F of the resin component 1, and thus the defective part 1F cannot be detected.

Next, requirements imposed on the camera 3 will be described.

The camera 3 includes an area sensor, has such a focal length that the defective part 1F of the resin component 1 and the surrounding area can be imaged, and has effective pixels not less than one million pixels (1000 pixels×1000 pixels). The camera 3 is configured such that the focus can be manually adjusted and the F number, the shutter speed, and the ISO sensitivity can be arbitrarily set.

Next, requirements imposed on the blackout screen 11 will be described.

When the resin component 1 is imaged by the camera 3 in an environment with ambient light, the blackout screen 11 is used in order to block ambient light. For blocking ambient light, the blackout screen 11 is overlaid so as to cover the entire resin component appearance inspection device 100. In FIG. 1, a part of the blackout screen 11 is shown as an example.

The blackout screen 11 is required to satisfy the following specifications regarding the light blocking effect, the tearing resistance, and the flame retardancy.
(1) Light blocking effect: First grade in JIS L 1055 (light blocking rate of 99.99% or higher).
(2) Tearing resistance: 15 N or greater in JIS K 6328, or 15 N or greater by method D in JIS L 1096.
(3) Flame retardancy: A flame retardancy test number issued by public interest incorporated foundation Japan Fire Retardant Association has been acquired.

The light blocking effect influences the performance of appearance inspection by the resin component appearance inspection device 100. The tearing resistance and the flame retardancy do not influence the performance of appearance inspection, but it is desirable that the tearing resistance and the flame retardancy satisfy the specifications as fireproof measures in practical operation.

A resin component appearance inspection method for performing appearance inspection for the resin component 1 using the resin component appearance inspection device 100 will be described with reference to FIG. 11 which is a flowchart of the resin component appearance inspection method, FIG. 12 which is a flowchart of image processing, and FIG. 13A to FIG. 13F which show examples of processed images in resin component appearance inspection.

First, flow of the entire process of the resin component appearance inspection method will be described with reference to the flowchart shown in FIG. 11.

Using the resin component appearance inspection device 100, the resin component 1 is placed at the component retention mechanism 2, illumination light is emitted to the resin component 1 by the illumination 4, and an image of the resin component 1 is taken by the camera 3. The taken image is subjected to image processing by the image processing device 5, whereby a quantitative value of an appearance quality of the resin component 1 is calculated and outputted.

In FIG. 11, the “component retention mechanism” is described as “retention mechanism”. Also in the following drawings, the “component retention mechanism” may be described as “retention mechanism”.

The resin component appearance inspection method includes steps 01 (S01) to 06 (S06).

In a resin component placement step (S01), the resin component 1 is placed at the component retention mechanism 2. In a position adjustment step (S02), the distances and the angles of the camera 3 and the illumination 4 relative to the resin component 1 are adjusted.

In an imaging step (S03), illumination light is emitted to the resin component 1 by the illumination 4, and an image of the resin component 1 is taken by the camera 3.

In an image acquisition step (S04), the image of the resin component 1 taken by the camera 3 is acquired into the image processing device 5.

In an image processing step (S05), image processing described later in detail is performed on the acquired image, and a quantitative value of an appearance quality of the resin component 1 is calculated.

In a quantitative value output step (S06), the quantitative value of the appearance quality of the resin component 1 calculated by performing calculation processing on the image in the image processing step (S05) is outputted. The image processing device 5 stores the data into an internal storage device and displays the data on a display device. At this time, for example, the data may be outputted to an external host system such as a data collection device or the like.

Next, specific processing contents of the image processing step (S05) will be described with reference to a flowchart shown in FIG. 12.

The processing in the image processing step (S05) after the image processing device 5 acquires the image in the image acquisition step (S04) and before the quantitative value of the appearance quality is outputted in the quantitative value output step (S06), includes steps 51 (S51) to 56 (S56).

In a trimming step (S51), since a part other than the appearance inspection target in the resin component 1 and a part of the resin component appearance inspection device 100 also appear in the image, the defective part 1F of the resin component 1 is trimmed.

In a smoothing step (S52) and a difference image generation step 53 (S53), processing for correcting the luminance distribution according to the distance from the part where the illumination 4 appears reflected (hereinafter, referred to as a luminance distribution based on the illumination 4) is performed.

In the smoothing step (S52), since the luminance distribution based on the defective part 1F of the resin component 1 arises in a narrower area than the luminance distribution based on the illumination 4, an image is smoothed, whereby the luminance distribution based on the defective part 1F of the resin component 1 is eliminated once, so that only the luminance distribution based on the illumination 4 is left.

In a difference image generation step (S53), a difference between the image in the trimming step (S51) and the image in the smoothing step (S52) is acquired, whereby, conversely, only the luminance distribution based on the illumination 4 is eliminated, so that the luminance distribution based on the defective part 1F of the resin component 1 is left. In acquisition of the difference, since the luminance is represented by a value in a 256-gradation range of 0 to 255, the difference is calculated so as to fall within the luminance value range (0 to 255) using Formula (1).

$$[ ( Image in step 51 - image in step 52 ) / 2 ] + 128 \qquad Formula ( 1 )$$

Here, since the image in the trimming step (S51) and the image in the smoothing step (S52) are similar in luminance, the average of luminance of the image in the difference image generation step (S53) is about 128.

In a binarization step (S54), the luminance distribution based on the illumination 4 is corrected and then binarized, whereby the number of gradation levels of luminance is decreased, thus making it easy to derive a quantitative value. A threshold for binarization is set at 128 which is around the average of luminance of the image in the difference image generation step (S53).

Since the defective part 1F exhibits higher luminance than the proper part, white pixels in the defective part 1F are connected to each other in the binarized image, so that a white pixel area having a large contour is formed. On the other hand, in the proper part, an area where the luminance slightly exceeds 128 and an area where the luminance does not exceed 128 when the difference is acquired in the difference image generation step (S53) are subjected to binarization in the binarization step (S54), so that a white pixel area having a small contour is formed.

Instead of consecutively performing three steps which are the smoothing step (S52), the difference image generation step (S53), and the binarization step (S54), “adaptive threshold processing” of performing binarization processing while determining a threshold in accordance with a luminance value in a filter for a pixel may be used so that the above steps are integrated into one step.

The adaptive threshold processing has an advantage that the calculation load can be reduced by integration of the steps. However, there is a disadvantage that a determination criterion for determining a filter size for the adaptive threshold processing is absent and the filter size is likely to be set arbitrarily.

On the other hand, in the method of separating the processing into the smoothing step (S52), the difference image generation step (S53), and the binarization step (S54), a filter size is set by looking at an image outputted in the smoothing step (S52) so that the pattern of an appearance defect in the output image disappears. Thus, there is an advantage that a determination criterion for determining the filter size is clear and the filter size is less likely to be set arbitrarily.

In a contour extraction step (S55), the contour of the defective part 1F is detected. By extracting only a contour having a reference area or larger, only the defective part 1F is extracted.

In a quantitative value calculation step (S56), since the defective part 1F has been converted into white pixels and the proper part has been converted into black pixels through the process until the contour extraction step (S55), the ratio of white pixels, that is, the area ratio of the defective part to the entire image, is calculated by Formula (2), and the calculated value is used as a quantitative value representing the appearance quality of the resin component 1.

$$Ratio of white pixels = number of white pixels / total number of pixels \qquad Formula ( 2 )$$

By calculating the quantitative value of the appearance quality of the resin component 1, a feature of becoming darker as becoming farther from the part where the illumination 4 appears reflected and a feature that the defective part 1F is brighter than an area around the defective part 1F can be separated in the area around the part where the illumination 4 appears reflected.

Next, examples of processed images in the steps of image processing performed by the image processing device 5 will be described with reference to FIG. 13A to FIG. 13F.

The image in FIG. 13A is an image before image processing, acquired by the image processing device 5 in the image acquisition step (S04).

The image in FIG. 13B is an image processed in the trimming step (S51).

The image in FIG. 13C is an image processed in the smoothing step (S52).

The image in FIG. 13D is an image processed in the difference image generation step (S53).

The image in FIG. 13E is an image processed in the binarization step (S54).

The image in FIG. 13F is an image processed in the contour extraction step (S55).

Next, regarding two kinds of appearance defects, examples of images before image processing by the image processing device 5 and images after the image processing will be described with reference to FIG. 14 and FIG. 15.

FIG. 14 and FIG. 15 show original images before image processing, images after image processing, and quantitative values of appearance qualities, in a case where the resin component 1 is a proper product and a case where the resin component 1 is a defective product. In the original image, a dotted-line rectangle at the upper right indicates a range trimmed in the trimming step (S51).

FIG. 14 shows an appearance defect called a tiger stripe in the field of injection molding, and a pattern of a plurality of streaks appears.

FIG. 15 shows an appearance defect called a flow mark in the field of injection molding, and an arc-shaped or sector-shaped pattern appears around an inflow port for resin.

In both of FIG. 14 and FIG. 15, the defective parts have been successfully detected as white pixels, and the quantitative values (white pixel ratios) are greater for defective products. By providing a threshold between quantitative values for a defective product and a proper product, proper-defective determination can be performed.

Here, the resin component appearance inspection method using the resin component appearance inspection device 100 will be summarized on the basis of the image processing performed by the image processing device 5.

The resin component appearance inspection method is performed using the component retention mechanism 2 which retains the resin component 1 which is an inspection target, the camera 3 which takes an image of the resin component 1, the illumination 4 which emits illumination light to the resin component 1, and the image processing device 5 which processes the image.

The resin component appearance inspection method includes step 04 (S04) and steps 51 (S51) to 56 (S56).

In the image acquisition step (S04), an image taken by the camera 3 is acquired into the image processing device 5. In the trimming step (S51), the defective part 1F of the resin component 1 is trimmed.

In the smoothing step (S52), the image is smoothed.

In the difference image generation step (S53), an image representing a difference between the image in the trimming step and the image in the smoothing step is generated.

In the binarization step (S54), an image in the difference image generation step is binarized.

In the contour extraction step (S55), only a contour of the defective part 1F is extracted.

In the quantitative value calculation step (S56), the pixel ratio of the defective part 1F, that is, a white pixel ratio, is calculated.

In the resin component appearance inspection device and the resin component appearance inspection method according to embodiment 1, without using machine learning, a defective part is converted into white pixels and a proper part is converted into black pixels, through image processing such as smoothing and contour detection, whereby a quantitative value is calculated. Therefore, images needed for resin component appearance inspection are only at least two images (border samples of a proper product and a defective product).

In the resin component appearance inspection device and the resin component appearance inspection method according to embodiment 1, a flow mark, a tiger stripe, and the like can be detected as a difference in luminance by using a difference between angle distributions of reflection light intensities for a defective part and a proper part. Therefore, it is possible to perform quantification even for an appearance defect that is not due to a macroscopic recess/projection.

As described above, the resin component appearance inspection device and the resin component appearance inspection method according to embodiment 1 can perform quantification even for an appearance defect that has a low contrast and is not due to a macroscopic recess/projection, while the number of images needed for inspection is small.

### Embodiment 2

In a resin component appearance inspection device and a resin component appearance inspection method according to embodiment 2, a driving device for moving a component retention mechanism is provided so that appearance inspection can be performed for an entire resin component while the resin component is moved, even in a case where the resin component has a long size.

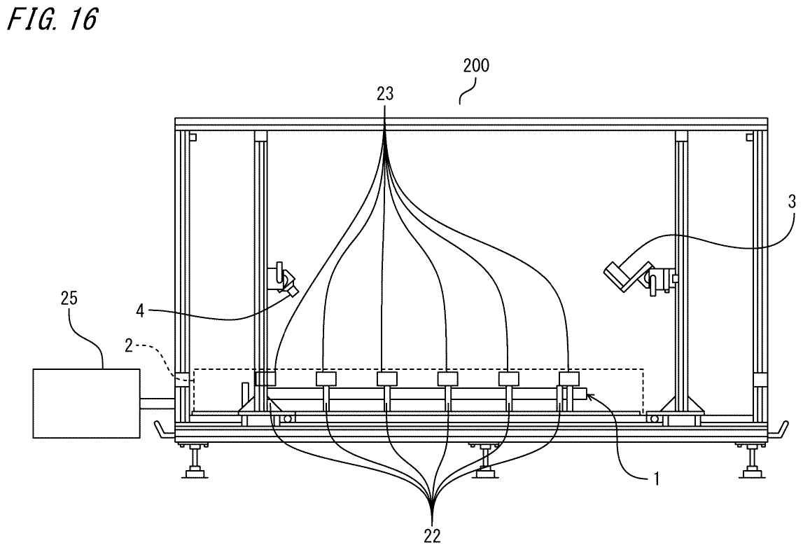

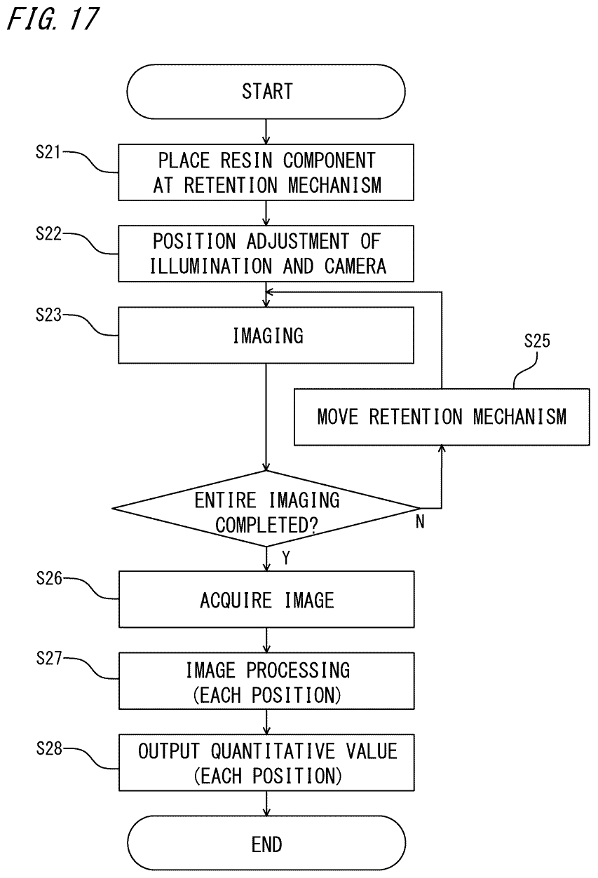

The resin component appearance inspection device and the resin component appearance inspection method according to embodiment 2 will be described focusing on a difference from embodiment 1 with reference to FIG. 16 which shows a structure of the resin component appearance inspection device, and FIG. 17 which is a flowchart of the resin component appearance inspection method.

In the drawings in embodiment 2, the same or corresponding parts as those in embodiment 1 are denoted by the same reference characters. For discrimination from embodiment 1, the resin component appearance inspection device is denoted by 200.

The entire structure of the resin component appearance inspection device 200 according to embodiment 2 will be described with reference to FIG. 16 which shows a structure of the inspection device.

The resin component appearance inspection device 200 includes, as main constituent parts, the resin component 1 which is an inspection target, the component retention mechanism 2 which retains the resin component 1, the camera 3 and the illumination 4 for taking an image of the resin component 1, the image processing device 5 which performs calculation processing on the taken image, the framework 10 which is a frame structure of the entire inspection device, and a driving device 25 which moves the component retention mechanism 2.

A difference from the resin component appearance inspection device 100 according to embodiment 1 is that the driving device 25 including a motor and the like for horizontally moving the component retention mechanism 2 with the resin component 1 placed thereon is added. In FIG. 16, the image processing device 5 and the blackout screen 11 are not shown.

The resin component 1 which is an inspection target has a flat-plate shape as in embodiment 1.

The structure of the component retention mechanism 2 and the specifications of the camera 3 and the illumination 4 are the same as those in embodiment 1.

Also, the setting method for the positional relationship of the resin component 1, the camera 3, and the illumination 4 is the same as that in embodiment 1.

In embodiment 1, it is assumed that appearance inspection for the resin component 1 is performed through visual recognition or the like in advance to specify a part where an appearance defect is likely to arise, and presence/absence of the defective part is inspected. Therefore, in performing appearance inspection, the position of the resin component 1 relative to the illumination 4 and the camera 3 is adjusted so that inspection for the defective part specified in advance can be performed with high accuracy.

In the resin component appearance inspection device 200 according to embodiment 2, such appearance inspection in advance is not needed, and it is possible to perform appearance inspection for the entire resin component 1 by taking images a plurality of times while moving the resin component 1, even in a case where the resin component 1 has a long size. However, adjustment of the positions and the angles of the illumination 4 and the camera 3, and the trimming position, is needed, and with their positional relationship kept, only the resin component 1 is moved in a horizontal direction, whereby appearance inspection for the entire resin component 1 is achieved.

The resin component 1 is placed at the component retention mechanism 2, illumination light is emitted to the resin component 1 by the illumination 4, and an image of the resin component 1 is taken by the camera 3. The component retention mechanism 2 with the resin component 1 placed thereon is moved a predetermined distance by the driving device 25, and then an image of a next part of the resin component 1 is taken by the camera 3. Through repetition of the above operation, images of the entire resin component 1 are taken, and the image processing described in FIG. 11 is performed on each of the taken images by the image processing device 5. As a result, appearance inspection for the entire resin component 1 can be performed.

The resin component appearance inspection method according to embodiment 2 includes steps 21 (S21) to 28 (S28). In a resin component placement step (S21), the resin component 1 is placed at the component retention mechanism 2. In a position adjustment step (S22), the distances and the angles of the camera 3 and the illumination 4 relative to the resin component 1 are adjusted.

In an imaging step (S23), illumination light is emitted to the resin component 1 by the illumination 4, and an image of the resin component 1 is taken by the camera 3.

In a completion determination step (S24), whether or not imaging of the entire resin component 1 has been completed is determined. If imaging of the entire resin component 1 has been completed, the process proceeds to a next image acquisition step (S26), and if imaging of the entire resin component 1 has not been completed, the process proceeds to a resin component movement step (S25).

In the resin component movement step (S25), the component retention mechanism 2 (i.e., the resin component 1) is moved a predetermined distance by the driving device 25. After the resin component movement step (S25), the process returns to the imaging step (S23).

In the image acquisition step (S26), the image of the resin component 1 taken by the camera 3 is acquired into the image processing device 5.

In an image processing step (S27), the image processing described above in detail is performed on each of the acquired images, and a quantitative value of an appearance quality of the resin component 1 is calculated.

In a quantitative value output step (S28), the quantitative value of the appearance quality of the resin component 1 calculated by performing calculation processing on the images in the image processing step (S27) is outputted. The image processing device 5 stores the data into an internal storage device and displays the data on a display device. At this time, for example, the data may be outputted to an external host system such as a data collection device or the like.

The predetermined distance the resin component 1 is moved in the resin component movement step (S24) is set to be approximately equal to the length in the slide movement direction of the defective part 1F that can be covered by one image. The slide movement distance (i.e., a distance in which the defective part 1F can be detected by one time of imaging) is about 10 cm to 15 cm.

When imaging is performed at each position of the resin component 1, each image may be labelled, whereby it becomes easy to specify a defect location in a case where an appearance quality inspection result by the image processing device 5 indicates that there is a defect.

Further, for each position (i.e., for each image) of the resin component 1 at which imaging is performed, a threshold may be provided between quantitative values for a defective product and a proper product, whereby appropriate proper-defective determination can be performed. For example, in a case where there are three defective parts 1F in the resin component 1, it is possible to set an appropriate threshold for each of the defective parts.

As described above, in the resin component appearance inspection device and the resin component appearance inspection method according to embodiment 2, the driving device for moving the component retention mechanism is provided so that appearance inspection can be performed for an entire resin component while the resin component is moved, even in a case where the resin component has a long size.

Therefore, the resin component appearance inspection device and the resin component appearance inspection method according to embodiment 2 can perform quantification even for an appearance defect that has a low contrast and is not due to a macroscopic recess/projection, while the number of images needed for inspection is small. Further, the entire resin component can be inspected.

### Embodiment 3

In a resin component appearance inspection device and a resin component appearance inspection method according to embodiment 3, a plurality of cameras and a plurality of illuminations are provided and the plurality of cameras and the plurality of illuminations are switched, whereby appearance inspection can be performed over the entire range even for a resin component having a long size.

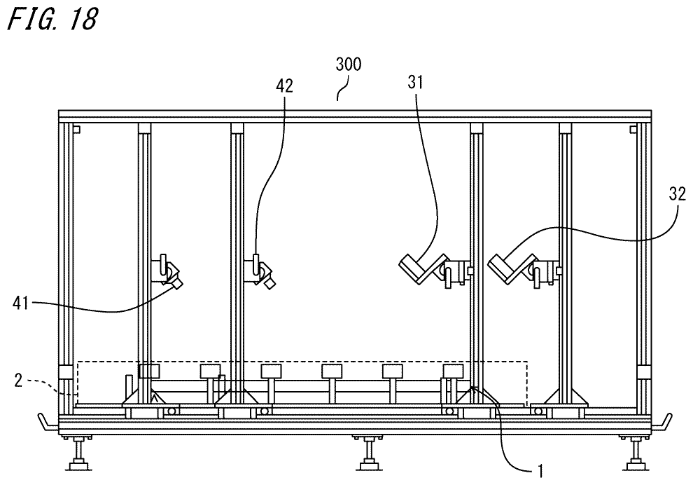

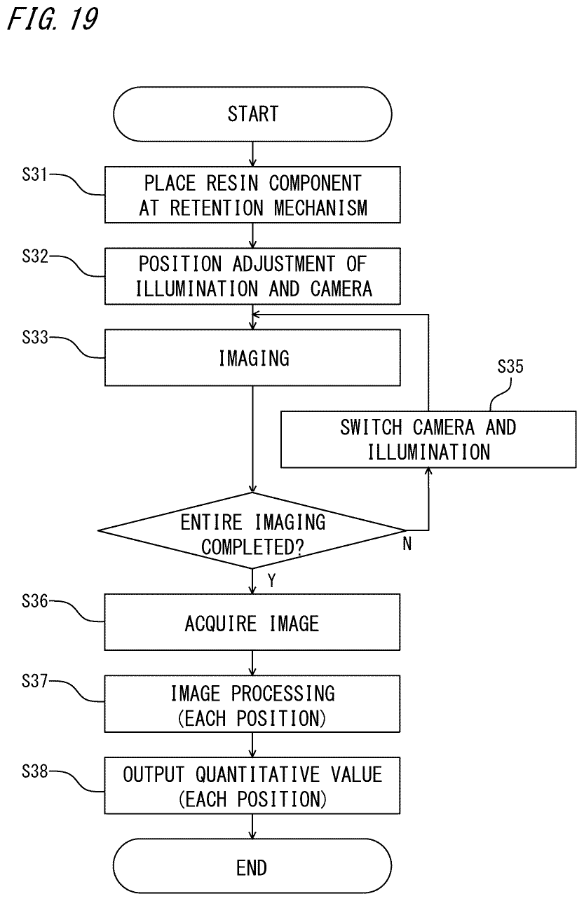

The resin component appearance inspection device and the resin component appearance inspection method according to embodiment 3 will be described focusing on a difference from embodiment 1 with reference to FIG. 18 which shows a structure of the resin component appearance inspection device and FIG. 19 which is a flowchart of the resin component appearance inspection method.

In the drawings in embodiment 3, the same or corresponding parts as those in embodiment 1 are denoted by the same reference characters. For discrimination from embodiment 1, the resin component appearance inspection device is denoted by 300.

The entire structure of the resin component appearance inspection device 300 according to embodiment 3 will be described with reference to FIG. 18 which shows a structure of the inspection device.

The resin component appearance inspection device 300 includes, as main constituent parts, the resin component 1 which is an inspection target, the component retention mechanism 2 which retains the resin component 1, two cameras 31 and 32 and two illuminations 41 and 42 for taking an image of the resin component 1, the image processing device 5 which performs calculation processing on the taken image, and the framework 10 which is a frame structure of the entire inspection device. A difference from the resin component appearance inspection device 100 according to embodiment 1 is that two cameras 31 and 32 and two illuminations 41 and 42 are provided. In FIG. 18, the blackout screen 11 is not shown.

In embodiment 3, a case where two cameras and two illuminations are provided will be described, but three or more cameras and three or more illuminations may be provided as appropriate in accordance with the dimensions of the resin component 1.

The resin component 1 which is an inspection target has a flat-plate shape as in embodiment 1.

The structure of the component retention mechanism 2 and the specifications of the camera 3 and the illumination 4 are the same as those in embodiment 1. Also, the setting method for the positional relationship of the resin component 1, the camera 3, and the illumination 4 is the same as that in embodiment 1.

In embodiment 1, it is assumed that appearance inspection for the resin component 1 is performed through visual recognition or the like in advance to specify a part where an appearance defect is likely to arise, and presence/absence of the defective part is inspected. Therefore, in performing appearance inspection, the position of the resin component 1 relative to the illumination 4 and the camera 3 is adjusted so that inspection for the defective part specified in advance can be performed with high accuracy.

In the resin component appearance inspection device 300 according to embodiment 3, such appearance inspection in advance is not needed, and it is possible to perform appearance inspection for the entire resin component 1 by taking images a plurality of times while switching the plurality of cameras and the plurality of illuminations in accordance with the dimensions of the resin component 1, even in a case where the resin component 1 has a long size. However, adjustment of the positions and the angles of the illuminations and the cameras, and the trimming position, is needed.

The resin component 1 is placed at the component retention mechanism 2, illumination light is emitted to a first part of the resin component 1 by the illumination 41, and an image of the first part of the resin component 1 is taken by the camera 31.

Next, the camera is switched to the camera 32 and the illumination is switched to the illumination 42, illumination light is emitted to a next part of the resin component 1 by the illumination 41, and an image of the next part of the resin component 1 is taken by the camera 32.

In a case where three or more cameras and three or more illuminations are provided, the above operation is repeated to take images for the entire resin component 1, and the image processing described in FIG. 11 is performed for each of the taken images by the image processing device 5. As a result, appearance inspection for the entire resin component 1 can be performed.

The resin component appearance inspection method according to embodiment 3 includes steps 31 (S31) to 38 (S38). In a resin component placement step (S31), the resin component 1 is placed at the component retention mechanism 2. In a position adjustment step (S32), the distances and the angles of the camera 31, 32 and the illumination 41, 42 relative to the resin component 1 are adjusted.

In an imaging step (S33), illumination light is emitted to the resin component 1 by the illumination 41, and an image of the resin component 1 is taken by the camera 31.

In a completion determination step (S34), whether or not imaging of the entire resin component 1 has been completed is determined. If imaging of the entire resin component 1 has been completed, the process proceeds to a next image acquisition step (S36), and if imaging of the entire resin component 1 has not been completed, the process proceeds to a camera and illumination switching step (S35).

In the camera and illumination switching step (S35), the camera is switched to the camera 32, and the illumination is switched to the illumination 42. After the camera and illumination switching step (S35), the process returns to the imaging step (S33).

In the image acquisition step (S36), an image of the resin component 1 taken by the camera 31, 32 is acquired into the image processing device 5.

In an image processing step (S37), the image processing described above in detail is performed on each of the acquired images, and a quantitative value of an appearance quality of the resin component 1 is calculated.

In a quantitative value output step (S38), the quantitative value of the appearance quality of the resin component 1 calculated by performing calculation processing on the images in the image processing step (S37) is outputted. The image processing device 5 stores the data into an internal storage device and displays the data on a display device. At this time, for example, the data may be outputted to an external host system such as a data collection device or the like.

A distance that can be covered by one image taken using, for example, the camera 31 and the illumination 41 (i.e., a distance in which the defective part 1F can be detected by one time of imaging), is about 10 cm to 15 cm.

When the resin component 1 is imaged, each image may be labelled, whereby it becomes easy to specify a defect location in a case where an appearance quality inspection result by the image processing device 5 indicates that there is a defect. Further, for each position (i.e., for each image) of the resin component 1 at which imaging is performed, a threshold may be provided between quantitative values for a defective product and a proper product, whereby appropriate proper-defective determination can be performed.

As described above, in the resin component appearance inspection device and the resin component appearance inspection method according to embodiment 3, a plurality of cameras and a plurality of illuminations are provided, and the plurality of cameras and the plurality of illuminations are switched, whereby appearance inspection can be performed over the entire range even for a resin component having a long size.

Therefore, the resin component appearance inspection device and the resin component appearance inspection method according to embodiment 3 can perform quantification even for an appearance defect that has a low contrast and is not due to a macroscopic recess/projection, while the number of images needed for inspection is small. Further, the entire resin component can be inspected.

### Embodiment 4

In a resin component appearance inspection device and a resin component appearance inspection method according to embodiment 4, a mold of an injection molding device is used instead of the component retention mechanism used in the resin component appearance inspection device according to each of embodiments 1 to 3.

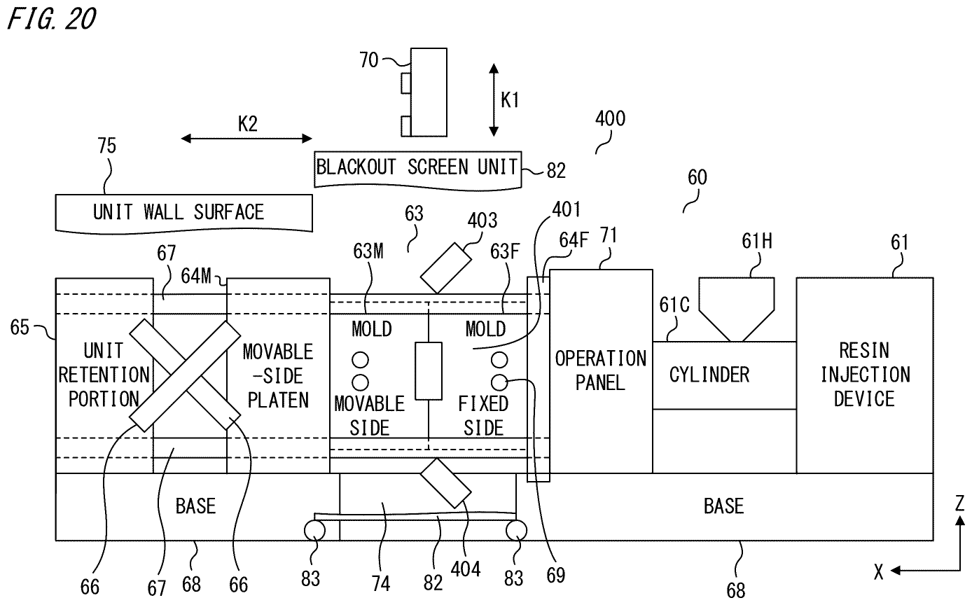

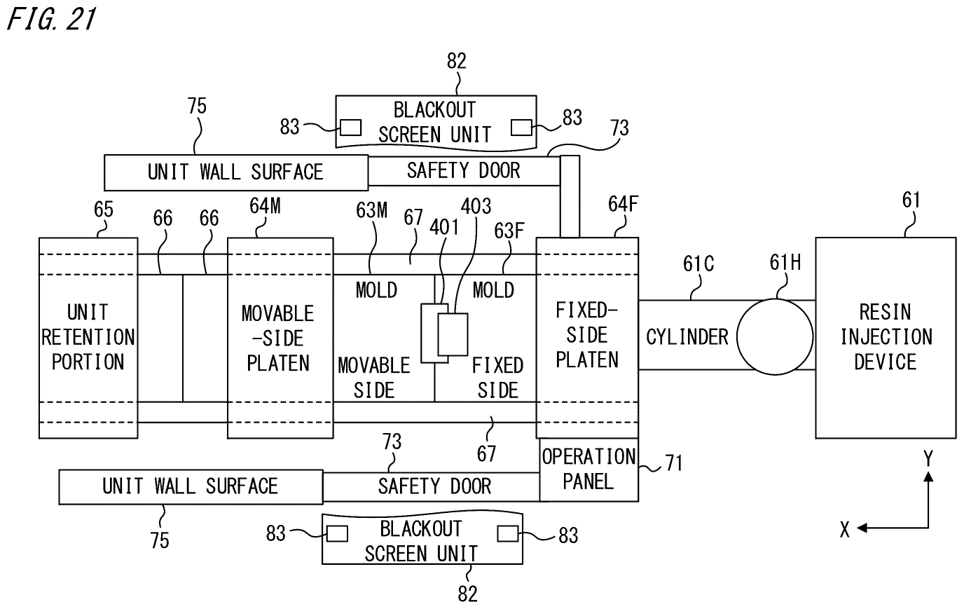

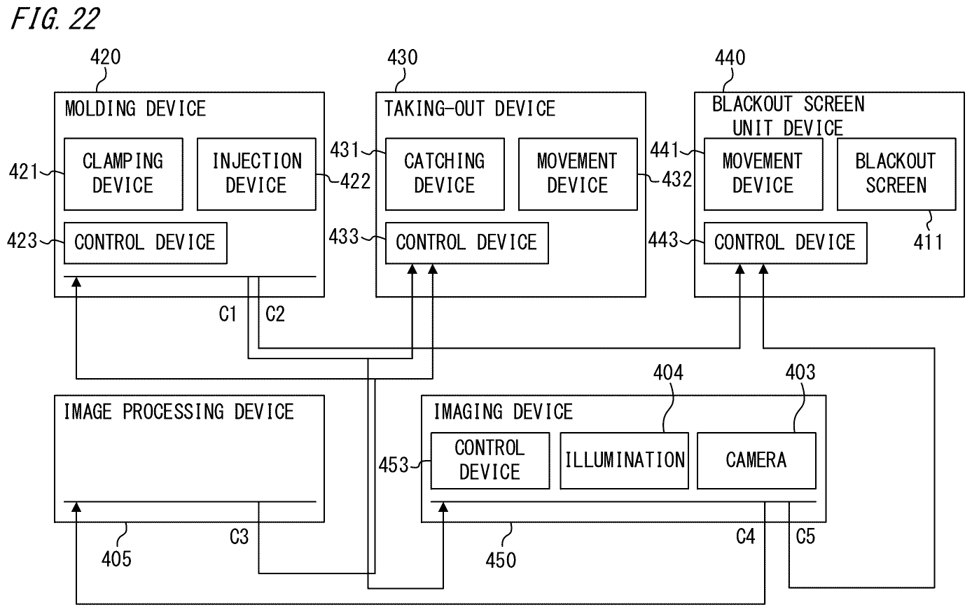

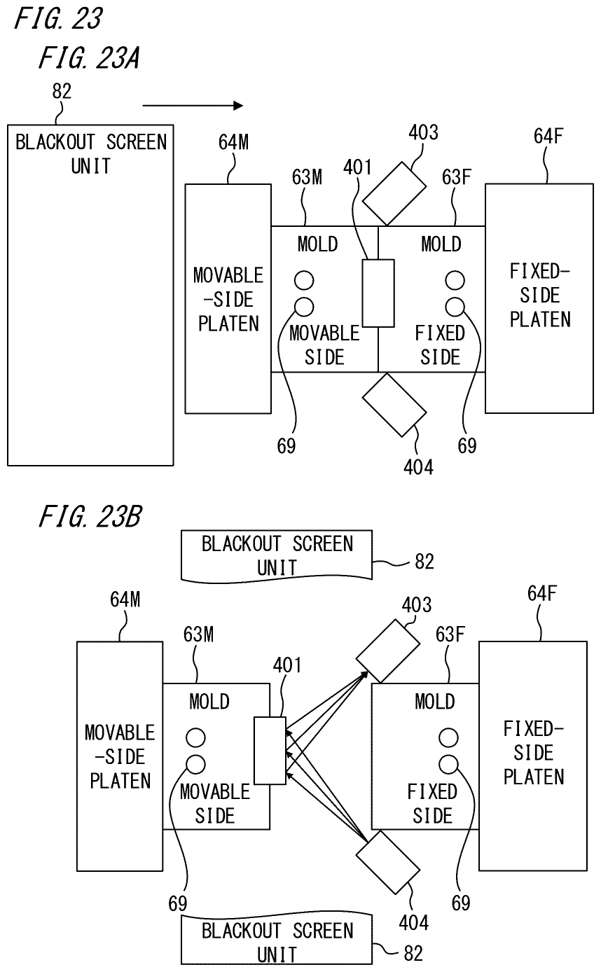

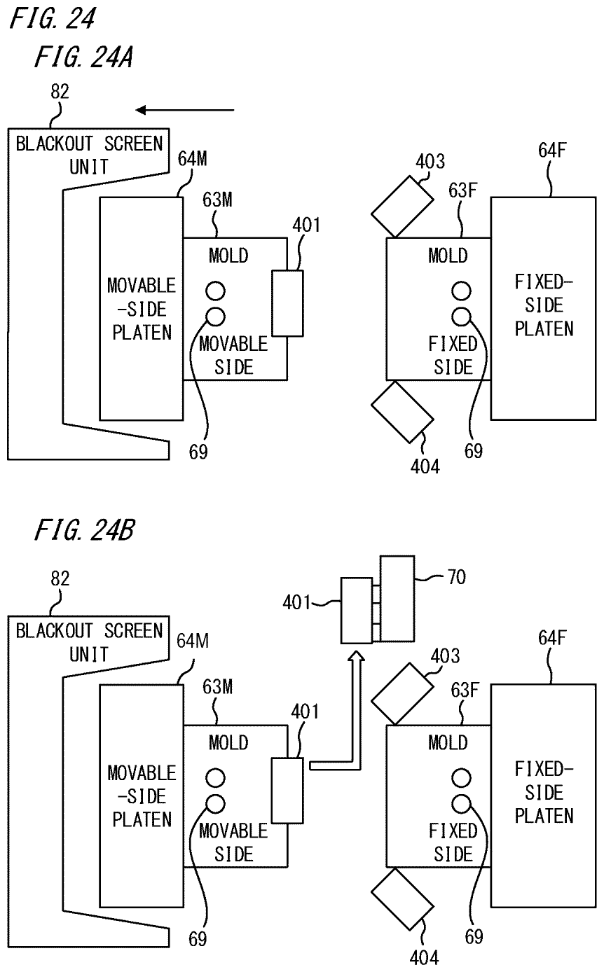

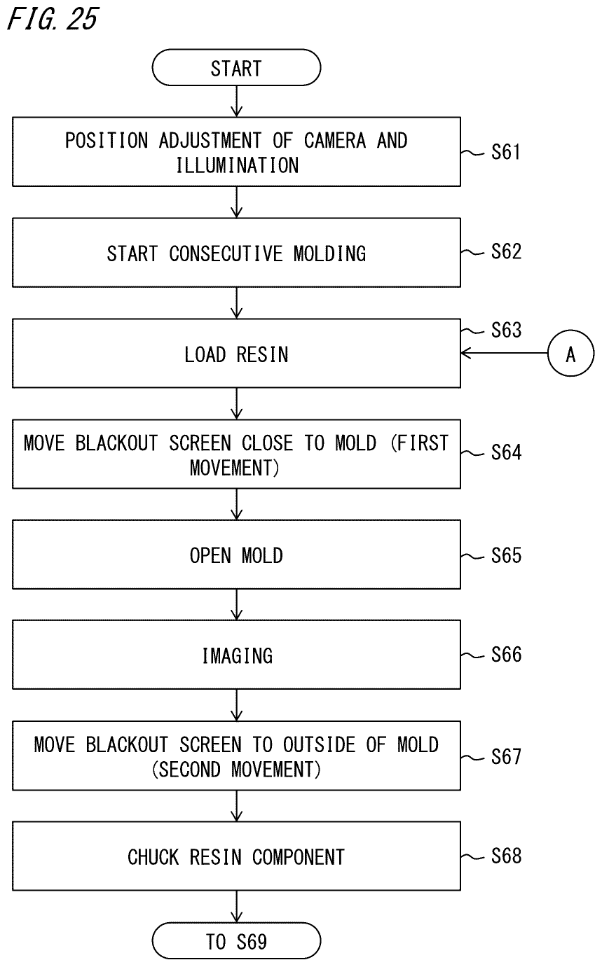

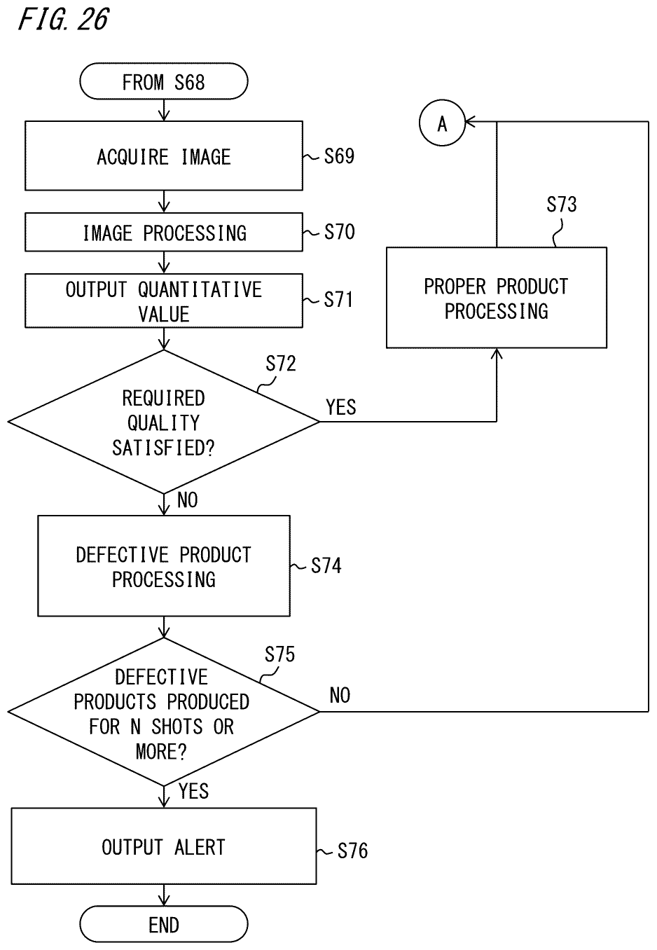

The resin component appearance inspection device and the resin component appearance inspection method according to embodiment 4 will be described focusing on a difference from embodiment 1 with reference to FIG. 20 and FIG. 21 which are system configuration diagrams of the resin component appearance inspection device, FIG. 22 which is a function block diagram of devices involved in the injection molding process, FIG. 23A, FIG. 23B, FIG. 24A, and FIG. 24B which illustrate an injection molding process, and FIG. 25 and FIG. 26 which are flowcharts of the resin component appearance inspection method.

In the drawings in embodiment 4, the same or corresponding parts as those in embodiment 1 are denoted by the same reference characters. For discrimination from embodiment 1, the resin component appearance inspection device is denoted by 400, the resin component is denoted by 401, the camera is denoted by 403, the illumination is denoted by 404, the image processing device is denoted by 405, and the blackout screen is denoted by 411.

In embodiments 1 to 3, an inspection target which is a molded product obtained by an injection molding device is referred to as a resin component. Although a component molded by a mold of an injection molding device may be referred to as a molded product, the molded product is referred to as a “resin component” also in embodiment 4, for the purpose of conformity with the descriptions in embodiments 1 to 3.

The system configuration of the resin component appearance inspection device 400 according to embodiment 4 will be described with reference to FIG. 20 and FIG. 21.

The resin component appearance inspection device 400 is configured such that members and devices such as the camera 403, the illumination 404, a blackout screen unit 82, and the image processing device 405, which are needed for appearance inspection for the resin component 401, are provided to the injection molding device 60.

FIG. 20 is a side view of the resin component appearance inspection device 400. In the drawing, the left-right direction is defined as an X direction, and the up-down direction is defined as a Z direction. A plus side in the Z direction (upper side in the drawing) is defined as a top side, and a minus side in the Z direction (lower side in the drawing) is defined as a bottom side.

FIG. 21 is a top view of the resin component appearance inspection device 400. In the drawing, the left-right direction is defined as an X direction, and the up-down direction is defined as a Y direction. The minus side in the Y direction (lower side in the drawing) is defined as an operation side, and the plus side in the Y direction (upper side in the drawing) is defined as a non-operation side.

In FIG. 20, K1 indicates movement of a taking-out machine 70 in accordance with progress of injection molding and appearance inspection for the resin component 401, and K2 indicates movement of the blackout screen unit 82.

In FIG. 20 and FIG. 21, a clamping unit retention portion 65 is described as a unit retention portion 65, and a clamping unit wall surface 75 is described as a unit wall surface 75.

First, a structure and a function of the injection molding device 60 will be described.

The injection molding device 60 includes a resin injection device 61, a clamping unit 62, and a mold 63. These devices are placed on a base 68. The injection molding device 60 further includes the taking-out machine 70 as a device for taking out the resin component 401 which is a product molded by a mold.

The resin injection device 61 is a device for heating and melting a plastic material and injecting the plastic material into the mold 63 in the injection molding process. A cylinder 61C which is one of main constituent parts of the resin injection device 61 serves to control supply and injection of a plastic material. A hopper 61H for providing a material is provided at an upper part of the resin injection device 61. The clamping unit 62 is a device for retaining and opening/closing the mold 63 for molding in the injection molding process, and includes a fixed-side platen 64F, a movable-side platen 64M, a clamping unit retention portion 65, a crosshead 66, and a tie bar 67.

When the fixed-side platen 64F and the movable-side platen 64M are collectively mentioned, they are referred to as platens 64.

The platens 64 are components that retain the mold 63 composed of a fixed-side die 63F and a movable-side die 63M and transmit a clamping force. The platens 64 are often manufactured by casting, and are designed so as not to be deformed by a clamping force.

When the fixed-side die 63F and the movable-side die 63M are collectively mentioned, they are referred to as the mold 63.

As the crosshead 66 operates, the mold 63 is opened/closed and the tie bar 67 is elastically deformed, so that a high-pressure clamping force arises. The clamping unit retention portion 65 retains the tie bar 67, and retains and drives the crosshead 66.

As a safety device, a safety door 73, a safety cover 74, and a clamping unit wall surface 75 are provided.

The safety door 73 is a device for protecting a working area of the injection molding device 60. Since the inside of the injection molding device 60 has a high temperature and a high pressure, the safety door 73 is provided for ensuring safety of an operator. A transparent material is used as a part of the safety door 73 so that the state of the mold 63 can be observed.

The safety cover 74 is provided on the bottom of the mold 63 so as to protect the working area of the injection molding device 60. As the safety cover 74, a metal plate is generally used, and therefore the inside cannot be seen. The clamping unit wall surface 75 is provided for safety and dust-proofing and is often formed by a metal plate.

An operation panel 71 is a device for operating and controlling the injection molding device 60, and is used for performing temperature control, injection control, and mold control.

The taking-out machine 70 is provided as a device needed in the injection molding process. The taking-out machine 70 takes out the resin component 401 after appearance inspection and moves the resin component 401 to predetermined places (a proper product place and a defective product place).

The blackout screen unit 82 needs to move along with advancement of the injection molding process, and therefore includes a movement device 83.

Next, the resin component appearance inspection device 400 will be described focusing on an appearance inspection method for the resin component 401.

The camera 403 and the illumination 404 are fixed to either the fixed-side die 63F or the movable-side die 63M. In FIG. 20 and FIG. 21, it is assumed that an ejection mechanism is provided to the movable-side die 63M (i.e., the resin component 401 is ejected from the movable side), and the camera 403 and the illumination 404 are fixed to the fixed-side die 63F, as an example. In a case where the resin component 401 is ejected from the fixed side, it is desirable that the camera 403 and the illumination 404 are fixed to the movable-side die 63M. In FIG. 20 and FIG. 21, it is assumed that a cooling pipe 69 of the mold 63 protrudes toward the operation side and the non-operation side, and the camera 403 and the illumination 404 are fixed on the top side and the bottom side of the mold 63. In a case where the cooling pipe 69 protrudes toward the top side and the bottom side, it is desirable that the camera 403 and the illumination 404 are fixed on the operation side and the non-operation side of the mold 63.

In FIG. 20 and FIG. 21, a case where the camera 403 and the illumination 404 are fixed to either the fixed-side die 63F or the movable-side die 63M is shown, but the camera 403 and the illumination 404 may be placed at either the fixed-side platen 64F or the movable-side platen 64M.

The blackout screen unit 82 includes a blackout screen 411 composed of a light blocking member and a support member, the movement device 83 composed of a caster, a motor, and the like, and a control device 443 for controlling the movement device 83. The blackout screen 411 blocks ambient light as with the blackout screen 11 shown in FIG. 1 in embodiment 1. In embodiment 4, in order to avoid interference with a movement line of the taking-out machine 70 of the resin component 401, the blackout screen unit 82 is retreated before the taking-out machine 70 approaches the resin component 401 that has undergone inspection.

Function blocks of the resin component appearance inspection device 400 according to embodiment 4 will be described with reference to FIG. 22. FIG. 22 shows functions of devices of the resin component appearance inspection device 400 by blocks, and these do not correspond one by one to hardware parts in the system configuration diagram shown in FIG. 20 and FIG. 21.

In FIG. 22, C1 indicates a “mold opening completion signal”, C2 indicates an “injection completion signal”, C3 indicates a “proper/defective product information”, C4 indicates “image data”, and C5 indicates an “imaging completion signal”.

A molding device 420 includes a clamping device 421, an injection device 422, and a control device 423.

The clamping device 421 opens or closes the mold 63, and the injection device 422 heats and melts a resin material and injects the resin material into the mold 63.

The control device 423 controls the clamping device 421 and the injection device 422.

A taking-out device 430 includes a chucking device 431, a movement device 432, and a control device 433.

The chucking device 431 chucks the resin component 401 by air suction or the like.

The movement device 432 moves the taking-out machine 70 close to the mold 63 after the mold 63 is opened, and moves the chucked resin component 401 to a proper/defective product place. When a mold opening completion signal (C1) is inputted from the molding device 420, the resin component 401 is moved to a proper product place or a defective product place on the basis of proper/defective product information (C3) from the image processing device 405.

The control device 433 controls the chucking device 431 and the movement device 432.

A blackout screen unit device 440 includes a movement device 441, a blackout screen 411, and a control device 443. The blackout screen 411 blocks ambient light as with the blackout screen 11 shown in FIG. 1 in embodiment 1. When an injection start signal (C2) is inputted from the molding device 420, the movement device 441 moves the blackout screen 411 close to the mold 63 on the basis of control by the control device 443.

When an imaging completion signal (C5) is inputted from an imaging device 450, the movement device 441 moves the blackout screen 411 to the outside of the mold 63 on the basis of control by the control device 443.

The imaging device 450 includes the camera 403, the illumination 404, and a control device 453. The functions of the camera 403 and the illumination 404 are the same as those in embodiment 1.

The control device 453 controls the camera 403 and the illumination 404. When a mold-opened signal (C1) is inputted from the molding device 420, the control device 453 lights up the illumination 404, releases the shutter of the camera 403, outputs image data (C4) to the image processing device 405, and turns off the illumination 404.

The image processing device 405 has the same function as the image processing device 5 in embodiment 1.

When the image data (C4) is inputted from the imaging device 450, the image processing device 405 performs image processing in the same manner as in embodiment 1, and outputs proper/defective product information (C3) to the molding device 420 and the taking-out device 430.

Next, operations of devices in embodiment 4, that is, operation of devices from loading of resin to taking out of a molded product, will be described with reference to FIG. 23A, FIG. 23B, FIG. 24A, and FIG. 24B.

FIG. 23A, FIG. 23B, FIG. 24A, and FIG. 24B show an example in which the cooling pipe 69 is provided in the top-bottom direction, the camera 403 is provided on the non-operation side of the fixed-side die 63F, and the illumination 404 is provided on the operation side of the fixed-side die 63F.

(1) In FIG. 23A, when loading of resin is started, the blackout screen 411 is moved close to the mold 63, so as to prepare for blocking ambient light at the time of imaging by the camera 403.

(2) In FIG. 23B, when loading of resin is completed, the mold 63 is opened, required illumination light is emitted to the resin component 401 by the illumination 404, and an image thereof is taken by the camera 403.

(3) In FIG. 24A, after imaging, the blackout screen 411 is moved to the outside of the mold 63, so as to prepare for avoiding interference when the resin component 401 is taken out by the taking-out machine 70.

(4) In FIG. 24B, after the blackout screen 411 is retreated, the resin component 401 is taken out by the taking-out machine 70.

Through repetition of the above operations (1) to (4), it is possible to consecutively perform molding while performing total inspection for appearance qualities of resin components 401.

FIG. 24B shows a state in which the taking-out machine 70 moves toward the non-operation side relative to the fixed-side die 63F, and the taking-out machine 70 also moves toward the top side relative to the fixed-side die 63F.

Next, flowcharts of the resin component appearance inspection method according to embodiment 4 will be described with reference to FIG. 25 and FIG. 26.

The resin component appearance inspection method according to embodiment 4 is a resin component appearance inspection method for performing appearance quality inspection for the resin component 401 using the injection molding device 60 which performs injection molding of the resin component 401 which is an inspection target. In this method, the camera 403 for taking an image of the resin component 401 and the illumination 404 for irradiating the resin component 401 are placed at the platen 64 or the mold 63 to be used for resin molding of the resin component 401, and the method is performed using the image processing device 405 which performs calculation processing on the image taken by the camera 403, the movable blackout screen 411, and the taking-out machine 70 which takes the resin component 401 out of the mold 63 and moves the resin component 401.

The resin component appearance inspection method includes steps 61 (S61) to 76 (S76).

In a position adjustment step (S61), the positions of the camera 403 and the illumination 404 are adjusted.

In a consecutive molding start step (S62), consecutive molding of the resin component 401 is started.

In a resin loading step (S63), resin is loaded into the mold 63 of the resin injection device 61.

In a first blackout screen movement step (S64), the blackout screen 411 is moved close to the mold 63.

In a mold opening step (S65), after loading of resin in the resin loading step (S63) is completed, the mold 63 is opened.

In an imaging step (S66), illumination light is emitted to the resin component 401 by the illumination 404, and an image of the resin component 401 is taken by the camera 403.

In a second blackout screen movement step (S67), the blackout screen 411 is moved to the outside of the mold 63. In a chucking step (S68), the taking-out machine 70 is moved close to the resin component 401, to chuck the resin component 401.

In an image acquisition step (S69), the image of the resin component 401 taken by the camera 403 is acquired into the image processing device 405.

In an image processing step (S70), the image processing described in embodiment 1 is performed on the acquired image, and a quantitative value of an appearance quality of the resin component 401 is calculated.

In a quantitative value output step (S71), the quantitative value of the appearance quality of the resin component 401 calculated by performing calculation processing in the image processing step (S70) is outputted. The image processing device 405 stores the data into an internal storage device and displays the data on a display device.

In a quality determination step (S72), the appearance quality of the resin component 401 outputted in the quantitative value output step (S71) is compared with a predetermined required quality, and whether or not the required quality is satisfied is determined. In the quality determination, evaluation may be performed using another quality item and another evaluation method, so as to be included as proper-defective determination.

In a proper product processing step (S73), if it is determined that the required quality is satisfied in the quality determination step (S72), the taking-out machine 70 moves the resin component 401 to a quality product place. Then, the process returns to the resin loading step (S63) to proceed to the next molding cycle.

In a defective product processing step (S74), if it is determined that the required quality is not satisfied in the quality determination step (S72), the taking-out machine 70 moves the resin component 401 to a defective product place. In a defect frequency determination step (S75), whether or not a defective product has been produced consecutively for a predetermined threshold number of shots (N shots) or more is determined. If a defective product has not been produced consecutively for N shots or more, the process returns to the resin loading step (S63) to proceed to the next molding cycle. If a defective product has been produced consecutively for N shots or more, the process proceeds to an alert output step (S76).

In the alert output step (S76), a consecutive defect alert is outputted and the consecutive molding process is stopped.

By performing a process through the resin component appearance inspection method including steps 61 (S61) to 76 (S76) described above, it is possible to consecutively perform molding while performing total inspection for appearance qualities of resin components 401. In a case where a defective product is produced, the defective product is discarded to a defective product place, and in a case where a defective product is produced consecutively, consecutive molding can be stopped. In a case where consecutive molding of resin components is stopped because a defect has occurred consecutively, a molding condition is adjusted so that the resin component 401 will become a proper product. Confirmation for whether or not a resin material lot has changed, confirmation of the state of the mold 63, and confirmation of change in environmental factors such as the ambient temperature and humidity, are performed. By performing necessary measures for a cause of occurrence of a defect, consecutive molding of the resin component 401 can be restored.

The effects of the resin component appearance inspection device and the molded product appearance inspection method according to embodiment 4 will be described.

In embodiment 4, since the mold of the injection molding device can be used as the resin component appearance inspection device, the resin component appearance inspection device need not be prepared as a separate device. In addition, it is possible to ensure reproducibility of a retention position for the resin component 401 having a warp and the resin component 401 having a projection such as a rib.

In the entire flowchart shown in FIG. 11 in embodiment 1, step S01 of “placing a resin component at the retention mechanism” is not needed, so that the number of steps in appearance inspection can be decreased. Further, the appearances of all molded products in consecutive molding can be automatically inspected in line, and therefore a defective product can be selected and can be automatically discarded by the taking-out machine. In addition, when a defective product is produced consecutively, an alert is issued and consecutive molding is stopped, whereby monitoring can be automated in mass production of injection molding products.

In the resin component appearance inspection device and the molded product appearance inspection method according to embodiment 4, it is assumed that a part where an appearance defect is likely to arise is specified in advance, and by providing a plurality of illuminations and a plurality of cameras as in embodiment 3, detection can be performed at a plurality of parts.

In addition, by controlling the angles of the camera 403 and the illumination 404 relative to the resin component 401, it is possible to perform appearance inspection for the entire resin component 401 while ensuring detection accuracy in a tolerable range.

As described above, in the resin component appearance inspection device and the molded product appearance inspection method according to embodiment 4, the mold of the injection molding device is used instead of the component retention mechanism used in the resin component appearance inspection device according to each of embodiments 1 to 3.

Therefore, the resin component appearance inspection device and the molded product appearance inspection method according to embodiment 4 can perform quantification even for an appearance defect that has a low contrast and is not due to a macroscopic recess/projection, while the number of images needed for inspection is small. Further, it is not necessary to prepare the resin component appearance inspection device separately.

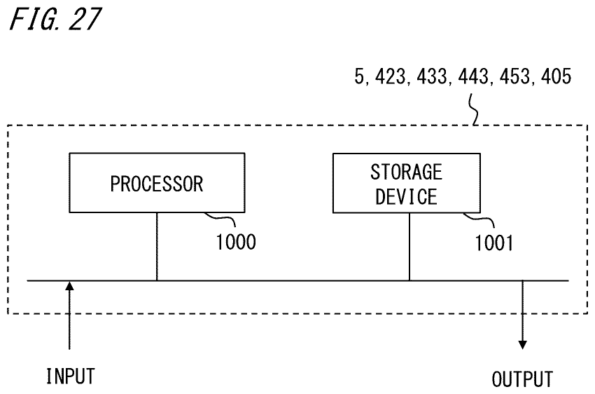

Here, an example of hardware of the image processing device 5, the control device 423 of the molding device 420, the control device 433 of the taking-out device 430, the control device 443 of the blackout screen unit device 440, the control device 453 of the imaging device 450, and the image processing device 405 in embodiments 1 to 4 will be described with reference to FIG. 27.

The image processing device 5, the control devices 423, 433, 443, and 453, and the image processing device 405 are composed of a processor 1000 and a storage device 1001. The storage device 1001 is provided with a volatile storage device such as a random access memory and a nonvolatile auxiliary storage device such as a flash memory, which are not shown. Instead of the flash memory, an auxiliary storage device of a hard disk may be provided. The processor 1000 executes a program inputted from the storage device 1001. In this case, the program is inputted from the auxiliary storage device to the processor 1000 via the volatile storage device. The processor 1000 may output data such as a calculation result to the volatile storage device of the storage device 1001, or may store such data into the auxiliary storage device via the volatile storage device.

Although the disclosure is described above in terms of various exemplary embodiments and implementations, it should be understood that the various features, aspects, and functionality described in one or more of the individual embodiments are not limited in their applicability to the particular embodiment with which they are described, but instead can be applied, alone or in various combinations to one or more of the embodiments of the disclosure.

It is therefore understood that numerous modifications which have not been exemplified can be devised without departing from the scope of the present disclosure. For example, at least one of the constituent components may be modified, added, or eliminated. At least one of the constituent components mentioned in at least one of the preferred embodiments may be selected and combined with the constituent components mentioned in another preferred embodiment.

Hereinafter, modes of the present disclosure are summarized as additional notes.

### Additional Note 1

A resin component appearance inspection device comprising:
a component retention mechanism which retains a resin component which is an inspection target;
a camera which takes an image of the resin component;
an illumination which irradiates the resin component; and
an image processing device which performs calculation processing on the image taken by the camera, wherein
the image processing device performs calculation processing on the image taken by the camera, to detect a defective part of a surface of the resin component, and quantifies an appearance quality of the resin component.

### Additional Note 2

The resin component appearance inspection device according to additional note 1, wherein
with respect to the image taken by the camera, the image processing device performs smoothing and acquisition of a difference, binarization and contour extraction of the defective part, calculation of a pixel ratio of the defective part, and calculation of a quantitative value of the appearance quality.

### Additional Note 3

The resin component appearance inspection device according to additional note 1 or 2, wherein
an angle formed by the illumination and the defective part of the resin component is defined as an incidence angle, and an angle formed by the camera and the defective part of the resin component is defined as a detection angle, and
the incidence angle is set at 35 degrees to 55 degrees, and the detection angle is provided with an angle difference of 3 degrees to 10 degrees relative to the incidence angle.

### Additional Note 4

The resin component appearance inspection device according to any one of additional notes 1 to 3, wherein
the illumination has a bar shape, emits light in a diffusing manner, and is a surface light emission type, the illumination has a luminance distribution characteristic that is highly uniform in a long-axis direction, and a length of a light emitting portion of the illumination is not smaller than two times a length of the defective part of the resin component.

### Additional Note 5

The resin component appearance inspection device according to any one of additional notes 1 to 4, wherein
the camera includes an area sensor and has effective pixels not less than one million pixels, and a focus, an F number, a shutter speed, and an ISO sensitivity of the camera are adjustable.

### Additional Note 6

The resin component appearance inspection device according to any one of additional notes 1 to 5, further comprising a driving device which moves the component retention mechanism, wherein
while the resin component is moved by the driving device, imaging of the resin component by the camera is repeated, to perform appearance inspection for an entirety of the resin component.

### Additional Note 7

The resin component appearance inspection device according to any one of additional notes 1 to 5, comprising a plurality of the cameras and a plurality of the illuminations, wherein
while the plurality of cameras and the plurality of illuminations are switched, imaging of the resin component by the camera is repeated, to perform appearance inspection for an entirety of the resin component by the cameras.

### Additional Note 8

The resin component appearance inspection device according to any one of additional notes 1 to 7, wherein
in a case where the resin component has a warp when manufactured, the warp has already been corrected at a time of appearance inspection.

### Additional Note 9

The resin component appearance inspection device according to any one of additional notes 1 to 8, further comprising a blackout screen which shields the camera and the illumination from ambient light.

### Additional Note 10

A resin component appearance inspection method using a component retention mechanism which retains a resin component which is an inspection target, a camera which takes an image of the resin component, an illumination which irradiates the resin component, and an image processing device which performs calculation processing on the image taken by the camera, the method comprising:
an image acquisition step of acquiring the image taken by the camera, into the image processing device;
a trimming step of trimming a defective part of a surface of the resin component;
a smoothing step of smoothing an image generated in the trimming step;
a difference image generation step of generating an image representing a difference between the image generated in the trimming step and an image generated in the smoothing step;
a binarization step of binarizing the image generated in the difference image generation step;
a contour extraction step of extracting a contour of the defective part; and
a quantitative value calculation step of calculating a pixel ratio of the defective part to perform quantification.

### Additional Note 11

The resin component appearance inspection method according to additional note 10, further using a driving device which moves the component retention mechanism, the method further comprising:
a resin component movement step of moving the resin component; and
a completion determination step of determining whether or not inspection for an entirety of the resin component has been completed.

### Additional Note 12

The resin component appearance inspection method according to additional note 10, using a plurality of the cameras and a plurality of the illuminations, the method further comprising:
a camera and illumination switching step of switching the plurality of cameras and the plurality of illuminations; and
a completion determination step of determining whether or not inspection for an entirety of the resin component has been completed.

### DESCRIPTION OF THE REFERENCE CHARACTERS

1, 401 resin component
2 component retention mechanism
3, 31, 32, 403 camera
4, 41, 42, 404 illumination
5, 405 image processing device
10 framework
11, 411 blackout screen
21 aluminum plate
22 knurled bolt
23 magnet
25 driving device
51 cable
100, 200, 300, 400 resin component appearance inspection device
60 injection molding device
61 resin injection device
61C cylinder
61H hopper
62 clamping unit
63 mold
63F fixed-side die
63M movable-side die
64 platen
64F fixed-side platen
64M movable-side platen
65 clamping unit retention portion
66 crosshead
67 tie bar
68 base
69 cooling pipe
70 taking-out machine
71 operation panel
73 safety door
74 safety cover
75 clamping unit wall surface
82 blackout screen unit
83 movement device
420 molding device
421 clamping device
422 injection device
423, 433, 443, 453 control device
430 taking-out device
431 chucking device
432 movement device
440 blackout screen unit device
441 movement device
450 imaging device
1000 processor
1001 storage device

1. A resin component appearance inspection device comprising:
a component retention mechanism which retains a resin component which is an inspection target;
a camera which takes an image of the resin component;
an illumination which irradiates the resin component; and
an image processing device which performs calculation processing on the image taken by the camera, wherein
the resin component is opaque and lustrous, and
the image processing device performs calculation processing on the image taken by the camera, to detect a defective part of a surface of the resin component, and quantifies an appearance quality of the resin component,
wherein
an angle formed by the illumination and the defective part of the resin component is defined as an incidence angle, and an angle formed by the camera and the defective part of the resin component is defined as a detection angle,
when the illumination emits illumination light to the resin component that is lustrous, reflection with the incidence angle and a reflection angle equal to each other is defined as specular reflection light, and a part where an appearance of the illumination appears reflected on the surface of the resin component is defined as regular reflection,
a part where reflection occurs also at angles different from the incidence angle due to a face that surfaces of a proper part and the defective part are not perfect mirror surfaces is defined as irregular reflection,
using a face that angle distributions of light intensities of the regular reflection and light intensities of the irregular reflection are different between the proper part and the defective part,
the detection angle is provided with an angle difference relative to the incidence angle,
so that a regular reflection light part of the spectacular reflection light is removed by trimming and an irregular reflection light part is extracted, and
thus, appearance defects of the resin component are detected.

2. The resin component appearance inspection device according to claim 1, wherein
with respect to the image taken by the camera, the image processing device performs smoothing and acquisition of a difference, binarization and contour extraction of the defective part, calculation of a pixel ratio of the defective part, and calculation of a quantitative value of the appearance quality, so that, in an area around a part where the illumination appears reflected, a feature of becoming darker as becoming farther from the part where the illumination appears reflected and a feature that the defective part is brighter than an area around the defective part, are separated.

3. The resin component appearance inspection device according to claim 1, wherein
the detection angle is provided with an angle difference of 3 degrees to 10 degrees relative to the incidence angle,
so that the irregular reflection light part is extracted, and
thus, a flow mark defect and a tiger stripe defect of the resin component recess/projection, are detected.

4. The resin component appearance inspection device according to claim 1, wherein
the illumination has a bar shape, emits light in a diffusing manner, and is a surface light emission type, the illumination has a luminance distribution characteristic that is highly uniform in a long-axis direction, and a length of a light emitting portion of the illumination is not smaller than two times a length of the defective part of the resin component.

5. The resin component appearance inspection device according to claim 1, wherein
the camera includes an area sensor and has effective pixels not less than one million pixels, and a focus, an F number, a shutter speed, and an ISO sensitivity of the camera are adjustable.

6. The resin component appearance inspection device according to claim 1, further comprising a driving device which moves the component retention mechanism, wherein
while the resin component is moved by the driving device, imaging of the resin component by the camera is repeated, to perform appearance inspection for an entirety of the resin component.

7. The resin component appearance inspection device according to claim 1, comprising a plurality of the cameras and a plurality of the illuminations, wherein
while the plurality of cameras and the plurality of illuminations are switched, imaging of the resin component by the camera is repeated, to perform appearance inspection for an entirety of the resin component by the cameras.

8. The resin component appearance inspection device according to claim 1, wherein
in a case where the resin component has a warp when manufactured, the warp has already been corrected at a time of appearance inspection.

9. The resin component appearance inspection device according to claim 1, further comprising a blackout screen which shields the camera and the illumination from ambient light.

10. The resin component appearance inspection device according to claim 1, wherein
the component retention mechanism includes an aluminum plate, a knurled bolt, and a magnet, and is configured such that
the resin component is fixed by being held between a head of the knurled bolt and the magnet, so that the resin component is retained in a state of being lifted up from a floor, and reproducibility of a retention position for the resin component having a warp and the resin component having a projection such as a rib is ensured.

11. The resin component appearance inspection device according to claim 1, wherein
the component retention mechanism is provided on a fixed side or a movable side where an ejection mechanism is present, of a mold used in a resin molding process for the resin component, and is configured such that
reproducibility of a retention position for the resin component having a warp and the resin component having a projection such as a rib is ensured.

12. A resin component appearance inspection method using a component retention mechanism which retains a resin component which is an inspection target, a camera which takes an image of the resin component, an illumination which irradiates the resin component, and an image processing device which performs calculation processing on the image taken by the camera,
wherein
an angle formed by the illumination and the defective part of the resin component is defined as an incidence angle, and an angle formed by the camera and the defective part of the resin components is defined as a detection angle,
when the illumination emits illumination light to the resin component that is lustrous, reflection with the incidence angle and a reflection angle equal to each other is defined as specular reflection light, and a part where an appearance of the illumination appears reflected on the surface of the resin component is defined as regular reflection,
a part where reflection occurs also at angles different from the incidence angle due to a fact that surfaces of the resin component are not perfect mirror surfaces is defined as irregular reflection,
the detection angle is provided with an angle difference relative to the incidence angle,
so that a regular reflection light part of the specular reflection light is removed by trimming and an irregular reflection light part is extracted,
the method comprising:
an image acquisition step of acquiring the image taken by the camera, into the image processing device;
a trimming step of trimming a defective part of a surface of the resin component;
a smoothing step of smoothing an image generated in the trimming step;
a difference image generation step of generating an image representing a difference between the image generated in the trimming step and an image generated in the smoothing step;
a binarization step of binarizing the image generated in the difference image generation step;
a contour extraction step of extracting a contour of the defective part; and
a quantitative value calculation step of calculating a pixel ratio of the defective part to perform quantification.

13. The resin component appearance inspection method according to claim 12, further using a driving device which moves the component retention mechanism, the method further comprising:
a resin component movement step of moving the resin component; and
a completion determination step of determining whether or not inspection for an entirety of the resin component has been completed.

14. The resin component appearance inspection method according to claim 12, using a plurality of the cameras and a plurality of the illuminations, the method further comprising:
a camera and illumination switching step of switching the plurality of cameras and the plurality of illuminations; and
a completion determination step of determining whether or not inspection for an entirety of the resin component has been completed.

15. A resin component appearance inspection method in which, using an injection molding device for performing injection molding of a resin component which is an inspection target, appearance quality inspection for the resin component is performed,
in a state in which a camera which takes an image of the resin component and an illumination which irradiates the resin component are placed at a mold or a platen to be used for resin molding of the resin component, the method being performed using an image processing device which performs calculation processing on the image taken by the camera, a movable blackout screen, and a taking-out machine which takes the resin component out of the mold and moves the resin component,
the method comprising:
a position adjustment step of adjusting positions of the camera and the illumination;
a consecutive molding start step of starting consecutive molding of the resin component;
a resin loading step of loading resin into the mold;
a first blackout screen movement step of moving the blackout screen close to the mold;
a mold opening step of opening the mold after loading of the resin in the resin loading step is completed;
an imaging step of emitting illumination light to the resin component by the illumination and taking an image of the resin component by the camera;
a second blackout screen movement step of moving the blackout screen to outside of the mold;
a chucking step of moving the taking-out machine close to the resin component and chucking the resin component;
an image acquisition step of acquiring the image of the resin component taken by the camera, into the image processing device;
an image processing step of performing image processing on the acquired image and calculating a quantitative value of an appearance quality of the resin component;
a quantitative value output step of outputting the quantitative value of the appearance quality of the resin component calculated in the image processing step;
a quality determination step of comparing the appearance quality of the resin component outputted in the quantitative value output step with a predetermined required quality, and determining whether or not the required quality is satisfied;
a proper product processing step of, in a case where it is determined that the required quality is satisfied in the quality determination step, moving the resin component to a proper product place by the taking-out machine and returning to the resin loading step;
a defective product processing step of, in a case where it is determined that the required quality is not satisfied in the quality determination step, moving the resin component to a defective product place by the taking-out machine;
a defect frequency determination step of determining whether or not a defective product has been produced consecutively a predetermined threshold number of times or more, and in a case where a defective product has not been produced consecutively the predetermined threshold number of times or more, returning to the resin loading step, and in a case where a defective product has been produced consecutively the predetermined threshold number of times or more, proceeding to an alert output step; and
the alert output step of outputting a consecutive defect alert and stopping a consecutive molding process.

16. The resin component appearance inspection device according to claim 2, wherein
the detection angle is provided with an angle difference of 3 degrees to 10 degrees relative to the incidence angle,
so that the irregular reflection light part is extracted, and
thus, a flow mark defect and a tiger stripe defect of the resin component are detected.

17. The resin component appearance inspection device according to claim 2, wherein
the illumination has a bar shape, emits light in a diffusing manner, and is a surface light emission type, the illumination has a luminance distribution characteristic that is highly uniform in a long-axis direction, and a length of a light emitting portion of the illumination is not smaller than two times a length of the defective part of the resin component.

18. The resin component appearance inspection device according to claim 2, wherein
the camera includes an area sensor and has effective pixels not less than one million pixels, and a focus, an F number, a shutter speed, and an ISO sensitivity of the camera are adjustable.

19. The resin component appearance inspection device according to claim 2, further comprising a driving device which moves the component retention mechanism, wherein
while the resin component is moved by the driving device, imaging of the resin component by the camera is repeated, to perform appearance inspection for an entirety of the resin component.

20. The resin component appearance inspection device according to claim 2, comprising a plurality of the cameras and a plurality of the illuminations, wherein
while the plurality of cameras and the plurality of illuminations are switched, imaging of the resin component by the camera is repeated, to perform appearance inspection for an entirety of the resin component by the cameras.
